(19) United States
(12) Patent Application Publication
(10) Pub. No.: US 2011/0319473 A1
(43) Pub. Date: Dec. 29, 2011

(54) COMPOSITIONS AND METHODS FOR ENHANCEMENT OF NUCLEIC ACID DELIVERY

(75) Inventors: Joseph Schmidt McGonigle, St. Paul, MN (US); Joram Slager, St. Louis Park, MN (US)

(73) Assignee: SURMODICS, INC., Eden Prairie, MN (US)

(21) Appl. No.: 13/171,171

(22) Filed: Jun. 28, 2011

Related U.S. Application Data
(60) Provisional application No. 61/359,814, filed on Jun. 29, 2010.

(51) Int. Cl. A61K 31/713; C07H 21/02

(52) U.S. Cl. 514/44 A; 536/24.5

(57) ABSTRACT

Embodiments of the invention include devices and methods for delivery of nucleic acids as active agents. Embodiments of the invention include devices and methods for delivery of nucleic acids as active agents. In an embodiment, an article for delivering an active agent is included. The article can include a dehydrated complex including a nucleic acid, a transfection agent, and a saccharide protectant. The nucleic acid and transfection agent can form a liposome or a lipoplex. The dehydrated complex can be disposed within a polymeric matrix. The dehydrated complex can be disposed within a microparticle. Other embodiments are also included herein.

This application claims the benefit of U.S. Provisional Application No. 61/359,814, filed Jun. 29, 2010, the contents of which are herein incorporated by reference.

### FIELD OF THE INVENTION

The present invention relates to devices and methods for delivery of active agents. More specifically, the present invention relates to devices and methods for delivery of nucleic acids as active agents.

### BACKGROUND OF THE INVENTION

One promising approach to the treatment of various medical conditions is the administration of nucleic acids as a therapeutic agent. However, successful treatment with nucleic acids can depend on various aspects including site-specific delivery, stability during the delivery phase, and a substantial degree of biological activity within target cells. For various reasons, these steps can be difficult to achieve.

One technique for administering nucleic acid based active agents is to use an implant as a delivery platform. The use of an implant for this purpose can provide site specific delivery of nucleic acids. However, there are numerous practical challenges associated with the use of such implants including manufacturing challenges, shelf stability, desirable elution profiles, sufficient active agent loading, and the like.

### SUMMARY OF THE INVENTION

Embodiments of the invention include devices and methods for delivery of nucleic acids as active agents. In an embodiment, an article for delivering an active agent is included. The article can include a dehydrated complex including a nucleic acid, a transfection agent, and a saccharide protectant. The nucleic acid and transfection agent can form a liposome or a lipoplex. The dehydrated complex can be disposed within a polymeric matrix. The dehydrated complex can be disposed within a microparticle.

In an embodiment, a method of maintaining the activity of a nucleic acid and transfection agent for incorporation in a controlled release formulation is included. The method can include combining a nucleic acid, a transfection agent, and a saccharide protectant in an aqueous solution to form an active agent composition. The method can also include removing water from the active agent composition to form dehydrated complexes. The method can further include resuspending the dehydrated complexes in a solution including an organic solvent. The method can further include forming microparticles from the dehydrated complexes and a polymeric composition.

In an embodiment, the invention includes a method of making a controlled release formulation. The method can include combining a nucleic acid, a transfection agent, and a saccharide in an aqueous solvent to form an active agent composition. The method can further include processing the active agent composition to remove the aqueous solvent and form dehydrated complexes. The method can further include combining the dehydrated complexes with a polymer composition. In some embodiments, the polymer composition can further include one or more organic solvent(s). The method can further include forming microparticles from the dehydrated complexes and the polymer composition.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope of the present invention is defined by the appended claims and their legal equivalents.

### BRIEF DESCRIPTION OF THE FIGURES

The invention may be more completely understood in connection with the following drawings, in which:

FIG. 1 is a schematic view of dehydrated complexes in accordance with various embodiments herein.

FIG. 2 is a schematic view of microparticles including dehydrated complexes in accordance with various embodiments herein.

FIG. 3 is a schematic view of a device in accordance with various embodiments herein.

FIG. 4 is a schematic view of a device in accordance with various embodiments herein.

FIG. 5 is a schematic view of a device in accordance with various embodiments herein.

FIG. 6 is a schematic view of a device in accordance with various embodiments herein.

FIG. 7 is a schematic view of a device in accordance with various embodiments herein.

FIG. 8 is a schematic view of a device in accordance with various embodiments herein.

FIG. 9 is a cross-sectional view of the medical device of FIG. 8, as taken along line 9-9′.

FIG. 10 is a graph of gene knock-down data for NTER/siRNA complexes.

FIG. 11 is a graph of gene knock-down data for DOTAP/siRNA liposomes.

FIGS. 12A-12B are graphs of gene knock-down data for siRNA/DOTAP complexes in the form of both liposomes and lipoplexes.

FIG. 13 is a graph of gene knock-down data for DOTAP/siRNA lipoplexes with various saccharide protectants.

FIG. 14 is a graph of gene knock-down data for DOTAP/siRNA lipoplexes.

FIG. 15 is a graph of siRNA release from microparticles including various polymers.

FIG. 16 is a graph of DOTAP release from microparticles including various polymers.

FIG. 17 is a graph showing the ratio of siRNA/DOTAP during release.

FIG. 18 is a graph of gene knock-down data for DOTAP/siRNA lipoplexes.

FIG. 19 is a graph of controlled release of siRNA from terpolymers.

FIG. 20 is a graph of controlled release of siRNA from organogels.

FIG. 21 is a graph of gene knock-down data for DOTAP/siRNA as released from various terpolymers.

FIG. 22 is a graph of gene knock-down data for DOTAP/siRNA as released from various organogels.

FIG. 23 is a graph of gene knock-down data for LipofectamineRNAiMax/siRNA including glycogen or dextrose as saccharide protectants.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

Designing and manufacturing devices and/or coatings to deliver nucleic acids as active agents results in various challenges. One significant challenge relates to formulating compositions to carry and release the active agent while maintaining sufficient therapeutic effect of the nucleic acids. Specifically, various processing steps commonly associated with manufacturing devices (such as solvent exposure, varying temperature exposure, solvent removal, incompatible component exposure, etc.) may result in inactivation or activity reduction of complexed nucleic acids and transfection agents.

Embodiments of the invention can include devices, articles, and/or coating that include a dehydrated complex, wherein one or more components of the dehydrated complex function to maintain activity of a nucleic acid active agent and transfection agent complex. As one example, the dehydrated complex can include a complex comprising a nucleic acid and a transfection agent, and a saccharide protectant. As shown herein, such formulations can be used to maintain and/or enhance the activity of the nucleic acid and transfection agent complex during processing steps.

By way of example, in some embodiments the nucleic acid and transfection agent complex can be formed in an aqueous solvent. In some cases, it may be desirable to remove water (dehydrate) from the complexes through lyophilization, vacuum drying, or the like. However, the process of dehydrating the complexes can result in attenuated activity of the complex of the nucleic acid and transfection agent. As shown below, embodiments included herein can be used to maintain and/or enhance the activity of the complex of nucleic acid and transfection agent in conjunction with dehydration steps.

As another example, dehydrated complexes may be resuspended in an organic solvent as part of processing. However, resuspension in an organic solvent may lead to attenuation of the activity of the complex of the nucleic acid and transfection agent. As shown below, formulations in accordance with the embodiments included herein can be used to maintain and/or enhance the activity the nucleic acid and transfection agent complex in conjunction with resuspension in non-aqueous solvents.

Another challenge relating to delivery of nucleic acids as active agents involves limits on active agent loading. It will be appreciated that certain types of drug delivery devices, including for example depots, microparticles, organogels, other injectables, and coated devices, may have practical limits on the amounts of components that can be included therewith. For example, based on the fact that many cardiovascular stents are designed for intravascular placement, there is a practical limit on the size of the device and therefore a practical limit on the amount of material that can be provided therewith. Some embodiments herein feature relatively low amounts of saccharide protectants to nucleic acids and transfection agent, while still exhibiting desirable activity retention characteristics. By way of example, in an embodiment, the wt./wt. ratio of saccharide protectant to the nucleic acid and transfection agent in a dehydrated complex is equal to or less than 5 to 1. This ratio can result in a relatively high loading of nucleic acids in the resulting product.

### Dehydrated Complexes

Dehydrated complexes used with embodiments herein can include a nucleic acid and a transfection agent. The transfection agent can be configured to promote intracellular delivery of the nucleic acid. Dehydrated complexes used with embodiments herein can also include a saccharide protectant. Examples of saccharide protectants are described in greater detail below. Referring now to FIG. 1, a plurality of dehydrated complexes 102 are shown in accordance with an embodiment herein. The dehydrated complexes 102 can include a nucleic acid, a transfection agent, and a saccharide protectant. The dehydrated complexes 102 can be formed in accordance with various methods described in greater detail below.

Saccharide protectants can include monosaccharides, disaccharides, trisaccharides, oligosaccharides, and polysaccharides. Polysaccharides can be linear or branched polysaccharides. Exemplary saccharide protectants can include but are not limited to dextrose, sucrose, maltose, mannose, trehalose, and the like. Exemplary saccharide protectants can further include, but are not limited to, polysaccharides including pentose, and/or hexose subunits, specifically including glucans such as glycogen and amylopectin, and dextrins including maltodextrins, fructose, mannose, galactose, and the like. Polysaccharides can also include gums such as pullulan, arabinose, galactan, etc.

Saccharide protectants can also include derivatives of polysaccharides. It will be appreciated that polysaccharides include a variety of functional groups that can serve as attachment points or can otherwise be chemically modified in order to alter characteristics of the saccharide. As just one example, it will be appreciated that saccharide backbones generally include substantial numbers of hydroxyl groups that can be utilized to derivatize the saccharide. By way of example, saccharides can be derivatized with hydrophobic pendent groups. Greater detail regarding derivatization with hydrophobic groups is discussed below. However, in some embodiments, where a polysaccharide with hydrophobic pendent groups is used as a saccharide protectant, the degree of substitution is less than 0.3.

Saccharide protectants can also include copolymers and/or terpolymers, and the like, that include saccharide and/or saccharide subunits and/or blocks.

Polysaccharides used with embodiments herein can have various molecular weights. By way of example, glycogen used with embodiments herein can have a molecular weight of greater than about 250,000. In some embodiments glycogen used with embodiments herein can have a molecular weight of between about 100,000 and 10,000,000 Daltons.

Refinement of the molecular weight of polysaccharides can be carried out using diafiltration. Diafiltration of polysaccharides such as maltodextrin can be carried out using ultrafiltration membranes with different pore sizes. As an example, use of one or more cassettes with molecular weight cut-off membranes in the range of about 1K to about 500 K can be used in a diafiltration process to provide polysaccharide preparations with average molecular weights in the range of less than 500 kDa, in the range of about 100 kDa to about 500 kDa, in the range of about 5 kDa to about 30 kDa, in the range of about 30 kDa to about 100 kDa, in the range of about 10 kDa to about 30 kDa, or in the range of about 1 kDa to about 10 kDa. It will be appreciated that polysaccharides such as maltodextrin and amylose of various molecular weights are commercially available from a number of different sources. For example, Glucidex™ 6 (ave. molecular weight˜95,000 Da) and Glucidex™ 2 (ave. molecular weight˜300,000 Da) are available from Roquette (France); and MALTRIN™ maltodextrins of various molecular weights, including molecular weights from about 12,000 Da to 15,000 Da are available from GPC (Muscatine, Iowa).

Nucleic acids used with embodiments of the invention can include various types of nucleic acids that can function to provide a therapeutic effect. Exemplary types of nucleic acids can include, but are not limited to, ribonucleic acids (RNA), deoxyribonucleic acids (DNA), small interfering RNA (siRNA), micro RNA (miRNA), piwi-interacting RNA (piRNA), short hairpin RNA (shRNA), antisense nucleic acids, aptamers, ribozymes, locked nucleic acids and catalytic DNA.

Exemplary transfection agents used with embodiments of the invention can include those compounds that can be complexed with nucleic acids in order to preserve the activity of the nucleic acid and transfection agent complexes during the manufacturing and delivery processes. Exemplary transfection agents can also include those that can promote intracellular delivery of the nucleic acid. As such, transfection agents can enhance therapeutic uses of nucleic acids as administered to subjects.

Exemplary classes of suitable transfection agents can include both cationic compounds (compounds having a net positive charge) and charge neutral compounds. By way of example, suitable transfection agents can include cationic and non-cationic polymers and cationic and non-cationic lipids. Exemplary cationic lipids can include, but are not limited to, 3β-[N-(N′,N′-dimethylaminoethane)-carbamoyl]cholesterol hydrochloride (DC-cholesterol); 1,2-dioleoyl-3-trimethylammonium-propane (DOTAP); dimethyldioctadecylammonium (DDAB); 1,2-dioleoyl-sn-glycero-3-ethylphosphocholine (EPC); 1,2-di-O-octadecenyl-3-trimethylammonium propane (DOTMA); 1,2-di-(9Z-octadecenoyl)-3-dimethylammonium-propane (DODAP); 1,2-dilinoleyloxy-3-dimethylaminopropane (DLinDMA) and derivatives thereof. Exemplary helper or fusogenic lipids can include, but are not limited to, 1,2-dioleoyl-sn-glycero-3-phosphoethanolamine (DOPE); cholesterol; 1,2-dioctadecanoyl-sn-glycero-3-phosphocholine (DSPC); 1,2-distearoyl-sn-glycero-3-phosphoethanolamine (DSPE). Other exemplary lipids can include, but are not limited to, lipidoids, atuplex formulations, and PEGylated forms of lipids described above. In some cases a mixture of lipids can be used form complexes.

Suitable transfection agents can also include polycation containing cyclodextrin, histones, cationized human serum albumin, aminopolysaccharides such as chitosan, peptides such as poly-L-lysine, poly-L-ornithine, and poly(4-hydroxy-L-proline ester, and polyamines such as polyethylenimine (PEI), polypropylenimine, polyamidoamine dendrimers, and poly(beta-aminoesters).

Transfection agents can also include peptides, such as those that include a nucleic acid binding domain and a nuclear localization domain in order to form a peptide-nucleic acid delivery construct. As used herein, the term “peptide” shall include any compound containing two or more amino-acid residues joined by amide bond(s) formed from the carboxyl group of one amino acid (residue) and the amino group of the next one. As such, peptides can include oligopeptides, polypeptides, proteins, and the like. It will be appreciated that many different peptides are contemplated herein. One exemplary peptide, known as MPG, is a 27 amino acid bipartite amphipathic peptide composed of a hydrophobic domain derived from HIV-1 gp41protein and a basic domain from the nuclear localization sequence (NLS) of SV40 large T antigen (commercially available as the N-TER Nanoparticle siRNA Transfection System from Sigma-Aldrich, St. Louis, Mo.). Another exemplary peptide, known as MPGΔ^{NLS}, is also a 27 amino acid bipartite amphipathic peptide. Other exemplary peptides can include poly-arginine fusion peptides. Still other exemplary peptides include those with protein transduction domains linked with a double-stranded RNA binding domain.

Other transfection agents can include solid nucleic acid lipid nanoparticles (SNALPs), liposomes, protein transduction domains, polyvinyl pyrrolidone (PVP), peptides (including oligopeptides, polypeptides, proteins), and the like. Additionally, transfection agents may also be conjugated to molecules which allow them to target specific cell types. Examples of targeting agents include antibodies and peptides which recognize and bind to specific cell surface molecules.

Dehydrated complexes can be formed from transfection agents and nucleic acids through various processes. In some cases, for example, a cationic transfection agent interacts with an anionic nucleic acid molecule and condenses into a compact, ordered complex. As such, in some embodiments, the nucleic acid can simply be contacted with the transfection agent in order to form a complex between the nucleic acid and the transfection agent.

Nucleic acids complexed with a transfection agent including a lipid, lipidoid, or other molecule of an amphipathic nature can exist in at least two structurally distinct forms, a lipoplex or a liposome.

As used herein, the term “lipoplex” shall refer to an artificial vesicle consisting of a micelle (an aggregate of amphipathic molecules oriented with hydrophobic moieties pointed inward and polar groups pointing outwards) or lipid bilayer, with siRNA coating the exterior and interfacing with the polar groups of the molecules that make up the micelle or lipid bilayer.

As used herein, the term “liposome” shall refer to an artificial vesicle consisting of a continuous bilayer or multibilayer of lipids enclosing some of the nucleic acid active agent within the liposome.

Although lipoplexes and liposomes can frequently include the same components, their substantially differing structure can result from the preparation techniques used. Formation techniques for liposomes can include passive encapsulation, ethanol drop encapsulation, encapsulation of nucleic acid in ethanol destabilized liposomes, reverse-phase evaporation encapsulation and spontaneous vesicle formation by ethanol dilution to form stable nucleic acid lipid particles (SNALPs) (techniques reviewed in MacLachlan I. Liposomal formulations for nucleic acid delivery. In: Crooke ST, editor. Antisense Drug Technology: Principles, Strategies, and Applications. Boca Raton (Fla.): CRC Press; 2008. p. 237-70).

In contrast, to form lipoplexes, typically a liposome or micelle is formed first, and then a nucleic acid such as siRNA is complexed to the outer surface of the liposome or micelle. For example, DOTAP (as merely one non-limiting example of a transfection agent) can first be formulated as micelles in distilled deionized water before reacting with siRNA. In one approach, the ethanol of the DOTAP solution is first evaporated on a rotovap forming a film of DOTAP. Then, using sonication, DOTAP is dissolved in distilled deionized water forming nano-size micelles. Subsequent reaction with siRNA can then form lipoplexes, where siRNA coats the outside of the micelles. It will be appreciated, however, that other approaches to the formation of lipoplexes and liposomes can be used.

In some embodiments, the amount of saccharide protectant is relatively small in comparison to the amount of nucleic acid and transfection agent. By way of example in some embodiments, the wt./wt. ratio of saccharide protectant to the nucleic acid and transfection agent is less than or equal to 25 to 1. In some embodiments, the wt./wt. ratio of saccharide protectant to the nucleic acid and transfection agent is less than or equal to 15 to 1. In some embodiments, the wt./wt. ratio of saccharide protectant to the nucleic acid and transfection agent is less than or equal to 10 to 1. In some embodiments, the wt./wt. ratio of saccharide protectant to the nucleic acid and transfection agent is less than or equal to 5 to 1.

It will be appreciated that formation of microparticles in accordance with embodiments herein can include various steps including one or more of contacting, combining, mixing, dispersing, sonicating, forming, extracting, removing solvents, drying, and the like. In some embodiments, a nucleic acid solution can be contacted with a transfection agent solution in order to form a combined reagent solution. In some embodiments, the transfection agent solution can be treated first so as to form micelles before being contacted with the nucleic acid solution. In some embodiments, a saccharide protectant can be added to the nucleic acid solution, to the transfection agent solution, or after combination to the combined reagent solution. In some embodiments, other components such as polymers and/or excipients can also be added to the nucleic acid solution, the transfection agent solution, or after combination to the combined reagent solution. After complexation between the nucleic acid and the transfection agent, the solution including the same can be referred to as a complex solution.

In some embodiments, water can be removed from the complex solution in order to form the dehydrated complexes. It will be appreciated that water removal can be accomplished in various ways. Possible solvent removal methods can include steps of filtration, centrifugation, vacuum concentration, evaporation, lyophilization, spray drying, and the like. In some embodiments, the complex solution can be lyophilized. Lyophilization techniques can include steps of reducing the temperature and putting the content under vacuum. Removing water can result in the formation of the dehydrated complex. In accordance with various embodiments herein, the activity of the nucleic acid and transfection agent complexes can be protected against the adverse effect that dehydration and associated processing may have on activity.

In some embodiments, further steps can be taken with the dehydrated complex in order to produce coatings, devices such as implants, and the like. By way of example, in some embodiments, the dehydrated complexes can be combined with a solution including one or more polymers. In some embodiments, the solution including one or more polymer can also include an organic solvent. In some cases the dehydrated complexes can be resuspended in an organic solvent before being combined with a polymer solution. In some embodiments, the dehydrated complexes can be resuspended in the polymer solution directly. In accordance with various embodiments herein, the activity of the nucleic acid and transfection agent complexes can be protected against the adverse effect that resuspension in an organic solvent may have on activity.

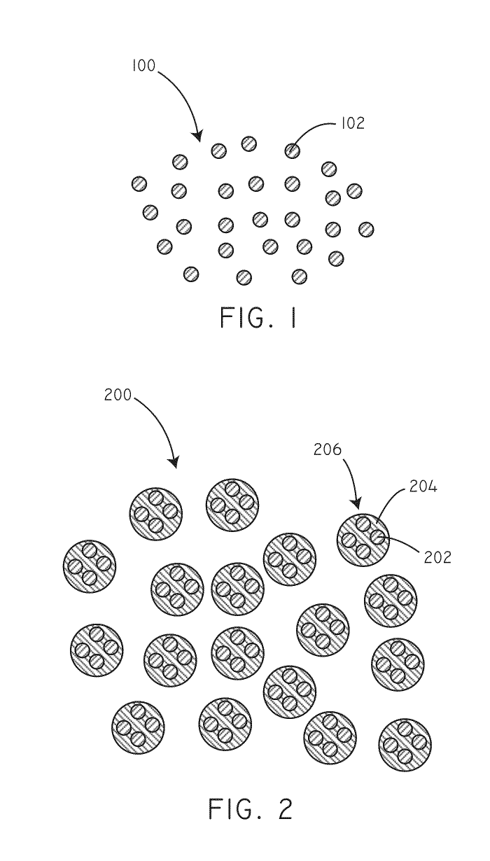

In some embodiments, the dehydrated complexes can be combined with a solution including one or more polymers and processed to form a particle (or microparticle) containing dehydrated complexes. Referring now to FIG. 2, a plurality of particles 206 are shown in accordance with an embodiment herein. Each particle 206 can include a one or more dehydrated complexes 202 disposed within a polymeric matrix 204.

It will be appreciated that various techniques can be used to form such particles (including microparticles). By way of example, the particle can be prepared by the process substantially as described in U.S. Pat. No. 5,407,609, herein incorporated by reference. The process can be an emulsion-based process which involves the preparation of an emulsion comprising an aqueous continuous phase (water and a surfactant and/or thickening agent) and a hydrophobic phase (polymer solvent, polymer and dehydrated complexes). Temperatures may be ambient, generally being from about 15 to 30° C. After formation of the emulsion, the polymer solvent can be extracted into an aqueous extraction phase. After a sufficient amount of polymer solvent is extracted to harden the microparticles, the microparticles can be collected on sieves and washed to remove any surfactant remaining on the surface of the microparticles. The microparticles can then dried with a nitrogen stream for an extended period, e.g. about 12 hours, then dried in a vacuum oven at room temperature until at least substantially dry, conveniently for about 3 days in some embodiments.

A relatively simple apparatus may be employed for the preparation of microparticles. Using storage containers for the different streams, tubing, three-way valves and a homogenizer, the system is readily assembled. In addition, various monitoring devices may be included, such as flow meters, temperature monitors, particle size monitors, etc. The organic solution can be introduced into a first tube connected to a three way valve, which connects to the aqueous continuous phase and to the homogenizer. By controlling the rate of flow of the two streams into the line connecting the homogenizer, the ratio of the two streams can be controlled, as well as the residence time in the homogenizer. The effluent from the homogenizer exits through a line which connects to a three-way valve through which the water stream is introduced. Again, the rate of flow ratio controls the amount of water to the homogenizer effluent stream. The residence time of the water extraction step can be controlled by the length of tubing and the rate of flow of the combined streams. The microparticles can then segregated by size by passing through two or more sieves which eliminates microparticles outside the desired range.

Particles containing dehydrated complexes in accordance with embodiments herein can have various dimensions based on the specific nature of the components thereof and methods used to form the same. In some embodiments, the particles can have an average diameter between about 1 μm and about 500 μm. In some embodiments, the particles can have an average diameter between about 10 μm and about 150 μm.

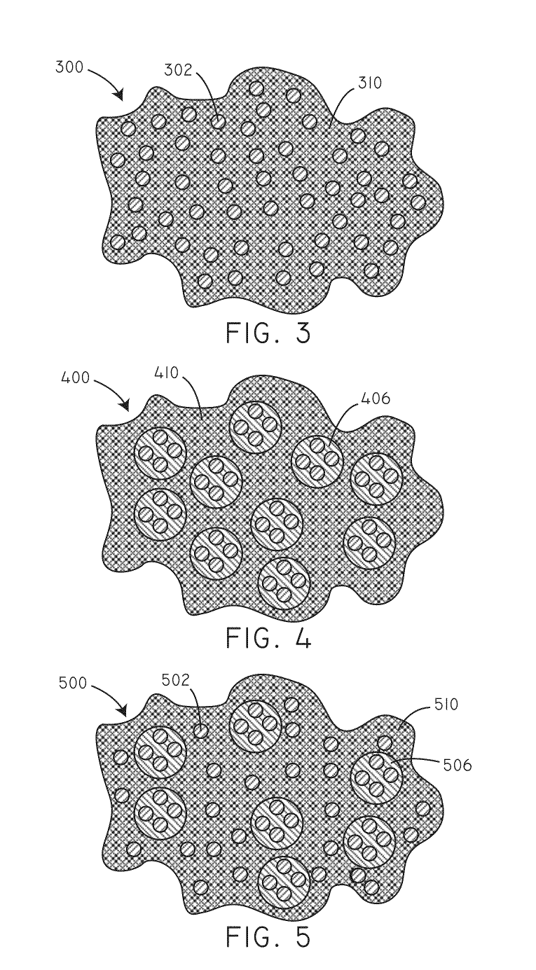

In some embodiments, the dehydrated complexes may be incorporated within a viscous liquid comprising a polymeric matrix. Referring now to FIG. 3, a device 300 (such as an implant) including a viscous polymeric matrix 310 is shown in accordance with an embodiment herein. As shown, dehydrated complexes 302 can be dispersed within the viscous polymer matrix 310. The polymers forming the polymeric matrix and having properties of a viscous liquid can include terpolymers. The terpolymer composition can be, in certain examples, a viscous, liquid-polymeric drug delivery platform capable of being administered by injection.

A terpolymer is a polymer comprised of three distinct monomer repeat units. Terpolymers can include those as described in U.S. Publ. Pat. App. No. 2009/0124535, the content of which is herein incorporated by reference. Terpolymers can include various distinct monomer repeat units such as lactide, glycolide, ε-caprolactone, amongst others. Some exemplary terpolymers can be prepared using (hydroxyl-containing) alcoholic initiators. The choice and selection of the alcoholic initiator allows one methods by which the attributes of the final polymer can be changed or manipulated. For example, the final viscosity of the resulting terpolymer can be affected by selection of a lipid-like or long-chain alcoholic initiators or a low-viscosity alcoholic initiator. For example, a terpolymer prepared using the lipid-like initiator 1-dodecanol or oleyl alcohol can have a lower viscosity than a similar terpolymer prepared from a small-molecular initiator (such as ethyl glycolate). Also, the relative lipophilicity of the resulting polymer can be affected by selection of a medium or long-chain (lipid-like) alcoholic initiator. The relative hydrophilicity of the resulting polymer can be affected by selection of a hydrophilic or a water-soluble alcoholic initiator (such as, for example, methoxy PEG-400). Hydrophobic initiators can be employed to slow down the relative degradation rate of a polymer while, conversely, a more hydrophilic initiator can result in a relatively faster degradation rate. The viscosity or rheological behavior of the resulting polymer can be affected by selection of a polymeric alcoholic initiator or by selection of an initiator containing two or more hydroxyl groups. Polyols can be used to prepare branched terpolymers having unusual rheological properties such as shear-thinning behavior or viscosities that are highly dependent on chain-length. Further, changes to the monomer composition allow additional routes by which the characteristics of the final polymer can be adjusted. For example, manipulations to the copolymer composition (such as increasing the relative caprolactone content, for example) can be utilized to lower the glass transition temperature, lower viscosity, alter hydrophobicity, and to affect manipulate overall degradation rates.

It will be appreciated that microparticles including dehydrated particles (as distinct from dehydrated particles alone), can also be incorporated within viscous polymeric matrices. Referring now to FIG. 4, a device 400 is shown including a viscous polymeric matrix 410 including a plurality of microparticles 406. The microparticles 406 can include one or more dehydrated complexes. In some embodiments, devices in accordance with embodiments herein can include both dehydrated complexes inside of microparticles in addition to dehydrated complexes outside of microparticles. Referring now to FIG. 5, an example is shown of a device 500 including a viscous polymeric matrix 510 along with microparticles 506 including one or more dehydrated complexes as well as dehydrated complexes 502 outside of the microparticles 506.

In some embodiments, dehydrated complexes and/or dehydrated complexes within microparticles can be disposed within organogels. An organogel, as used herein, is a combination of a biocompatible polymer dissolved in a biocompatible solvent system that is comprised of one or more organic solvents, at least one of which is not miscible with water. The liquid can be, for example, an organic solvent, including for example, but not limited to, benzyl benzoate, ethyl heptanoate, ethyl octanoate, DMSO, NMP, triacetin, glycerol tri butyrate, glycofurol, DMI, mineral oil, or vegetable oil.

Organogels can be prepared, in some embodiments, by immobilizing an organic phase into a three-dimensional network coming from the self-assembly of a low molecular weight gelator molecule. It will be appreciated that organogels can also be formed in other ways. It will be appreciated that there various organogels systems which can be used including tyrosine based organogels, lecithin organogels, sorbitan ester organogels, fatty acid organogels, polyethylene organogels, and the like.

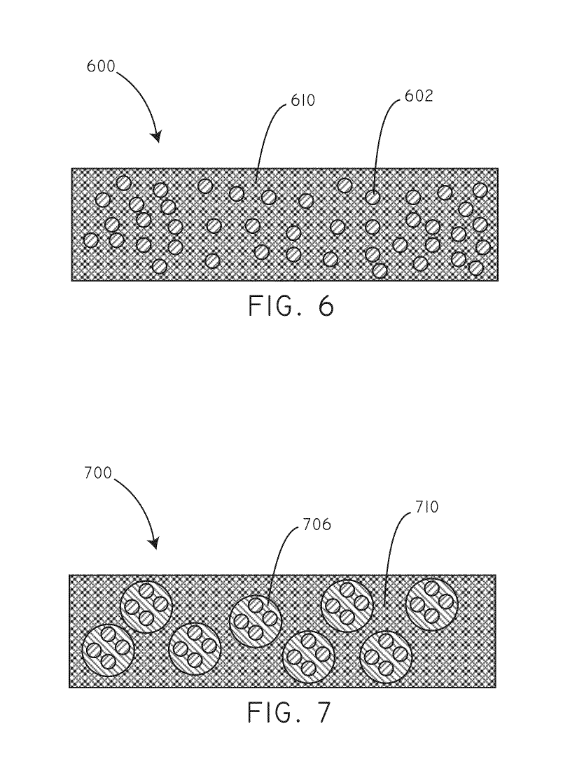

In some embodiments, dehydrated particles and/or microparticles including dehydrated particles can be incorporated within polymeric matrices that can include degradable polymers, non-degradable polymers, and/or combinations thereof. In some embodiments, dehydrated particles and/or microparticles can be included within polymeric matrices that are disposed on a substrate forming part of an implantable device. Referring now to FIG. 6, a schematic view is shown of a device 600 including a polymeric matrix 610. A plurality of dehydrated complexes 602 can be disposed within the polymeric matrix 610. Referring now to FIG. 7, a schematic view is shown of a device 700 including a polymer matrix 710. A plurality of microparticles 706 including dehydrated complexes disposed therein are within the polymeric matrix 710. It will be appreciated that polymeric matrices 610 and 710 can include various polymers including terpolymers, organogels, along with various other degradable and/or non-degradable polymers, or combinations thereof.

Further exemplary degradable polymers used with embodiments of the invention can include both natural or synthetic polymers. Examples of degradable polymers can include those with hydrolytically unstable linkages in the polymeric backbone. Examples of degradable polymers can also include those subject to enzymatic degradation. Degradable polymers of the invention can include both those with bulk erosion characteristics and those with surface erosion characteristics.

Synthetic degradable polymers can include: degradable polyesters (such as poly(glycolic acid), poly(lactic acid), poly(lactic-co-glycolic acid), poly(dioxanone), polylactones (e.g., poly(caprolactone)), poly(3-hydroxybutyrate), poly(3-hydroxyvalerate), poly(valerolactone), poly(tartronic acid), poly(β-malonic acid), polypropylene fumarate)); degradable polyesteramides; degradable polyanhydrides (such as poly(sebacic acid), poly(1,6-bis(carboxyphenoxy)hexane, poly(1,3-bis(carboxyphenoxy)propane); degradable polycarbonates (such as tyrosine-based polycarbonates); degradable polyiminocarbonates; degradable polyarylates (such as tyrosine-based polyarylates); degradable polyorthoesters; degradable polyurethanes; degradable polyphosphazenes; and copolymers thereof.

Degradable polyesters including those described above such as poly(glycolic acid) (PGA), poly(lactic acid) (PLA), poly(caprolactone) (PCL), poly(lactide-co-glycolide) (PLGA) amongst others can also be synthesized to contain blocks of polyethers (such as polyethylene glycol (PEG)) poloxamers (PLURONICS) as di- or tri-block-copolymers including, but not limited to, PLA-PEG-PLA, PGA-PEG-PGA, PLGA-PEG-PLGA, PEG-PLA, PEG-PGA, PEG-PLGA, methoxy PEG variants thereof, and rearrangements thereof. It will be appreciated that such multi-block polymers can be formed through various known techniques. The relative weight percentages of the components can be varied in order to result in a multi-block copolymer with desirable characteristics. In some embodiments the wt. % of PEG in the multi-block copolymer can be from about 0.1 wt. % to about 99 wt. %. Molecular weight of the PEG can include, but is not limited to, 100 Da to 200,000 Da. In some embodiments, the wt. % of the polyester components of the multi-block copolymer can be from about 0.1 wt. % to about 99 wt. %.

Specific examples of degradable polymers include poly(ether ester) multiblock copolymers based on poly(ethylene glycol) (PEG) and poly(butylene terephthalate) that can be described by the following general structure:

[—(OCH₂CH₂)ₙ—O—C(O)—C₆H₄—C(O)—]x[—O—(CH₂)₄—O—C(O)—C₆H₄—C(O)—]y,

where —C₆H₄— designates the divalent aromatic ring residue from each esterified molecule of terephthalic acid, n represents the number of ethylene oxide units in each hydrophilic PEG block, x represents the number of hydrophilic blocks in the copolymer, and y represents the number of hydrophobic blocks in the copolymer. The subscript “n” can be selected such that the molecular weight of the PEG block is between about 300 and about 4000. The block copolymer can be engineered to provide a wide array of physical characteristics (e.g., hydrophilicity, adherence, strength, malleability, degradability, durability, flexibility) and active agent release characteristics (e.g., through controlled polymer degradation and swelling) by varying the values of n, x and y in the copolymer structure. Such degradable polymers can specifically include those described in U.S. Pat. No. 5,980,948, the content of which is herein incorporated by reference in its entirety.

Degradable polymers of the invention can include multi-block copolymers, comprising at least two hydrolyzable segments derived from pre-polymers A and B, which segments are linked by a multi-functional chain-extender and are chosen from the pre-polymers A and B, and triblock copolymers ABA and BAB, wherein the multi-block copolymer is amorphous and has one or more glass transition temperatures (Tg) of at most 37° C. (Tg) at physiological (body) conditions. The pre-polymers A and B can be a hydrolyzable polyester, polyetherester, polycarbonate, polyestercarbonate, polyanhydride or copolymers thereof, derived from cyclic monomers such as lactide (L,D or L/D), glycolide, ε-caprolactone, δ-valerolactone, trimethylene carbonate, tetramethylene carbonate, 1,5-dioxepane-2-one, 1,4-dioxane-2-one (para-dioxanone) or cyclic anhydrides (oxepane-2,7-dione). The multi-functional chain-extender can specifically be a diisocyanate chain-extender, but can also be a diacid or diol compound. The composition of the pre-polymers may be chosen in such a way that the maximum glass transition temperature of the resulting copolymer is below 37° C. at body conditions. To fulfill the requirement of a Tg below 37° C., some of the above-mentioned monomers or combinations of monomers may be more preferred than others. This may by itself lower the Tg, or the pre-polymer is modified with a polyethylene glycol with sufficient molecular weight to lower the glass transition temperature of the copolymer. The degradable multi-block copolymers can include hydrolysable sequences being amorphous and the segments may be linked by a multifunctional chain-extender, the segments having different physical and degradation characteristics. For example, a multi-block co-polyester consisting of a glycolide-ε-caprolactone segment and a lactide-glycolide segment can be composed of two different polyester pre-polymers. By controlling the segment monomer composition, segment ratio and length, a variety of polymers with properties that can easily be tuned can be obtained. Such degradable multi-block copolymers can specifically include those described in U.S. Publ. App. No. 2007/0155906, the content of which is herein incorporated by reference in its entirety.

Degradable polyesteramides can include those formed from the monomers OH-x-OH, z, and COOH-y-COOH, wherein x is alkyl, y is alkyl, and z is leucine or phenylalanine. Such degradable polyesteramides can specifically include those described in U.S. Pat. No. 6,703,040, the content of which is herein incorporated by reference in its entirety.

Degradable polymeric materials can also be selected from: (a) non-peptide polyamino polymers; (b) polyiminocarbonates; (c) amino acid-derived polycarbonates and polyarylates; and (d) poly(alkylene oxide) polymers.

In an embodiment, the degradable polymeric material is composed of a non-peptide polyamino acid polymer. Exemplary non-peptide polyamino acid polymers are described, for example, in U.S. Pat. No. 4,638,045 (“Non-Peptide Polyamino Acid Bioerodible Polymers,” Jan. 20, 1987). Generally speaking, these polymeric materials are derived from monomers, including two or three amino acid units having one of the following two structures illustrated below:

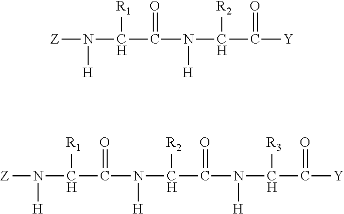

wherein the monomer units are joined via hydrolytically labile bonds at not less than one of the side groups R₁, R₂, and R₃, and where R₁, R₂, R₃ are the side chains of naturally occurring amino acids; Z is any desirable amine protecting group or hydrogen; and Y is any desirable carboxyl protecting group or hydroxyl. Each monomer unit comprises naturally occurring amino acids that are then polymerized as monomer units via linkages other than by the amide or “peptide” bond. The monomer units can be composed of two or three amino acids united through a peptide bond and thus comprise dipeptides or tripeptides. Regardless of the precise composition of the monomer unit, all are polymerized by hydrolytically labile bonds via their respective side chains rather than via the amino and carboxyl groups forming the amide bond typical of polypeptide chains. Such polymer compositions are nontoxic, are degradable, and can provide zero-order release kinetics for the delivery of active agents in a variety of therapeutic applications. According to these aspects, the amino acids are selected from naturally occurring L-alpha amino acids, including alanine, valine, leucine, isoleucine, proline, serine, threonine, aspartic acid, glutamic acid, asparagine, glutamine, lysine, hydroxylysine, arginine, hydroxyproline, methionine, cysteine, cystine, phenylalanine, tyrosine, tryptophan, histidine, citrulline, ornithine, lanthionine, hypoglycin A, β-alanine, γ-amino butyric acid, α aminoadipic acid, canavanine, venkolic acid, thiolhistidine, ergothionine, dihydroxyphenylalanine, and other amino acids well recognized and characterized in protein chemistry.

In some embodiments, degradable polymers used to form degradable matrices in accordance with embodiments herein can include a polysaccharide having one or more hydrophobic pendent groups attached to the polysaccharide. As described herein, the degree of substitution (DS) refers to the average number of reactive groups (including hydroxyl and other reactive groups) per monomeric unit that are substituted with pendent groups comprising hydrocarbon segments. In some embodiments, where a polysaccharide with hydrophobic pendent groups is used to form part of a degradable matrix in accordance with an embodiment herein, the degree of substitution is greater than equal to 0.3. In some embodiments, where a polysaccharide with hydrophobic pendent groups is used as a saccharide protectant, the degree of substitution is less than 0.3. Depending of course on the particular pendent group used for substitution, in many cases a degree of substitution of less than 0.3 will result in a modified polysaccharide that remains substantially water soluble.

Degradable polymers can also include natural degradable polysaccharide having one or more hydrophobic pendent groups attached to the polysaccharide. In many cases the hydrophobic derivative includes a plurality of groups that include hydrocarbon segments attached to the polysaccharide. When a plurality of groups including hydrocarbon segments are attached, they are collectively referred to as the “hydrophobic portion” of the hydrophobic derivative. The hydrophobic derivatives therefore include a hydrophobic portion and a polysaccharide portion.

The polysaccharide portion can include a natural degradable polysaccharide, which refers to a non-synthetic polysaccharide that is capable of being enzymatically degraded. Natural degradable polysaccharides include polysaccharide and/or polysaccharide derivatives that are obtained from natural sources, such as plants or animals. Natural degradable polysaccharides include any polysaccharide that has been processed or modified from a natural degradable polysaccharide (for example, maltodextrin is a natural degradable polysaccharide that is processed from starch). Exemplary natural degradable polysaccharides include maltodextrin, amylose, cyclodextrin, polyalditol, hyaluronic acid, dextran, heparin, chondroitin sulfate, dermatan sulfate, heparan sulfate, keratan sulfate, dextran, dextran sulfate, pentosan polysulfate, and chitosan. Specific polysaccharides are low molecular weight polymers that have little or no branching, such as those that are derived from and/or found in starch preparations, for example, maltodextrin, amylose, and cyclodextrin. Therefore, the natural degradable polysaccharide can be a substantially non-branched or completely non-branched poly(glucopyranose) polymer.

Another contemplated class of natural degradable polysaccharides is natural degradable non-reducing polysaccharides. A non-reducing polysaccharide can provide an inert matrix thereby improving the stability of active pharmaceutical ingredients (APIs), such as proteins and enzymes. A non-reducing polysaccharide refers to a polymer of non-reducing disaccharides (two monosaccharides linked through their anomeric centers) such as trehalose (α-D-glucopyranosyl α-D-glucopyranoside) and sucrose (β-D-fructofuranosyl α-D-glucopyranoside). An exemplary non-reducing polysaccharide includes polyalditol which is available from GPC (Muscatine, Iowa). In another aspect, the polysaccharide is a glucopyranosyl polymer, such as a polymer that includes repeating (1→3)O-β-D-glucopyranosyl units.

Dextran is an α-D-1,6-glucose-linked glucan with side-chains 1-3 linked to the backbone units of the dextran biopolymer. Dextran includes hydroxyl groups at the 2, 3, and 4 positions on the glucopyranose monomeric units. Dextran can be obtained from fermentation of sucrose-containing media by Leuconostoc mesenteroides B512F.

Dextran can be obtained in low molecular weight preparations. Enzymes (dextranases) from molds such as Penicillium and Verticillium have been shown to degrade dextran. Similarly many bacteria produce extracellular dextranases that split dextran into low molecular weight sugars.

Chondroitin sulfate includes the repeating disaccharide units of D-galactosamine and D-glucuronic acid, and typically contains between 15 to 150 of these repeating units. Chondroitinase AC cleaves chondroitin sulfates A and C, and chondroitin.

Hyaluronic acid (HA) is a naturally derived linear polymer that includes alternating β-1,4-glucuronic acid and β-1,3-N-acetyl-D-glucosamine units. HA is the principal glycosaminoglycan in connective tissue fluids. HA can be fragmented in the presence of hyaluronidase.

In many aspects the polysaccharide portion and the hydrophobic portion include the predominant portion of the hydrophobic derivative of the natural degradable polysaccharide. Based on a weight percentage, the polysaccharide portion can be about 25% wt of the hydrophobic derivative or greater, in the range of about 25% to about 75%, in the range of about 30% to about 70%, in the range of about 35% to about 65%, in the range of about 40% to about 60%, or in the range of about 45% to about 55%. Likewise, based on a weight percentage of the overall hydrophobic derivative, the hydrophobic portion can be about 25% wt of the hydrophobic derivative or greater, in the range of about 25% to about 75%, in the range of about 30% to about 70%, in the range of about 35% to about 65%, in the range of about 40% to about 60%, or in the range of about 45% to about 55%. In exemplary aspects, the hydrophobic derivative has approximately 50% of its weight attributable to the polysaccharide portion, and approximately 50% of its weight attributable to its hydrophobic portion.

The hydrophobic derivative has the properties of being insoluble in water. The term for insolubility is a standard term used in the art, and meaning 1 part solute per 10,000 parts or greater solvent. (see, for example, Remington: The Science and Practice of Pharmacy, 20th ed. (2000), Lippincott Williams & Wilkins, Baltimore Md.).

A hydrophobic derivative can be prepared by associating one or more hydrophobic compound(s) with a natural degradable polysaccharide polymer. Methods for preparing hydrophobic derivatives of natural degradable polysaccharides are described herein. In some embodiments, a “pendant group” can refer to a group of covalently bonded carbon atoms having the formula (CHₙ)ₘ, wherein m is 2 or greater, and n is independently 2 or 1. A hydrocarbon segment can include saturated hydrocarbon groups or unsaturated hydrocarbon groups, and examples thereof include alkyl, alkenyl, alkynyl, cyclic alkyl, cyclic alkenyl, aromatic hydrocarbon and aralkyl groups. Specifically, the pendant group includes linear, straight chain or branched C₁-C₂₀ alkyl group; an amine terminated hydrocarbon or a hydroxyl terminated hydrocarbon. In another embodiment, the pendant group includes polyesters such as polylactides, polyglycolides, poly(lactide-co-glycolide) co-polymers, polycaprolactone, terpolymers of poly(lactide-co-glycolide-co-caprolatone), or combinations thereof.

Various factors can be taken into consideration in the synthesis of the hydrophobic derivative of the natural degradable polysaccharide. These factors include the physical and chemical properties of the natural degradable polysaccharide, including its size, and the number and presence of reactive groups on the polysaccharide and solubility, the physical and chemical properties of the compound that includes the hydrocarbon segment, including its the size and solubility, and the reactivity of the compound with the polysaccharide.

In preparing the hydrophobic derivative of the natural degradable polysaccharide any suitable synthesis procedure can be performed. Synthesis can be carried out to provide a desired number of groups with hydrocarbon segments pendent from the polysaccharide backbone. The number and/or density of the pendent groups can be controlled, for example, by controlling the relative concentration of the compound that includes the hydrocarbon segment to the available reactive groups (e.g., hydroxyl groups) on the polysaccharide.

The type and amount of groups having the hydrocarbon segment pendent from the polysaccharide can be sufficient for the hydrophobic polysaccharide to be insoluble in water in some embodiments. In order to achieve this, as a general approach, a hydrophobic polysaccharide is obtained or prepared wherein the groups having the hydrocarbon segment pendent from the polysaccharide backbone in an amount in the range of 0.25 (pendent group): 1 (polysaccharide monomer) by weight.

The weight ratio of glucopyranose units to pendent groups can vary, but will typically be about 1:1 to about 100:1. Specifically, the weight ratio of glucopyranose units to pendent groups can be about 1:1 to about 75:1, or about 1:1 to about 50:1. Additionally, the nature and amount of the pendent group can provide a suitable degree of substitution to the polysaccharide. Typically, the degree of substitution will be in the range of about 0.1-5 or about 0.5-2.

To exemplify these levels of derivation, very low molecular weight (less than 10,000 Da) glucopyranose polymers are reacted with compounds having the hydrocarbon segment to provide low molecular weight hydrophobic glucopyranose polymers. In one mode of practice, the natural degradable polysaccharide maltodextrin in an amount of 10 g (MW 3000-5000 Da; ˜3 mmols) is dissolved in a suitable solvent, such as tetrahydrofuran. Next, a solution having butyric anhydride in an amount of 18 g (0.11 mols) is added to the maltodextrin solution. The reaction is allowed to proceed, effectively forming pendent butyrate groups on the pyranose rings of the maltodextrin polymer. This level of derivation results in a degree of substitution (DS) of butyrate group of the hydroxyl groups on the maltodextrin of about 1.

For maltodextrin and other polysaccharides that include three hydroxyl groups per monomeric unit, on average, one of the three hydroxyl groups per glycopyranose monomeric unit becomes substituted with a butyrate group. A maltodextrin polymer having this level of substitution is referred to herein as maltodextrin-butyrate DS 1.

An increase in the DS can be achieved by incrementally increasing the amount of compound that provides the hydrocarbon segment to the polysaccharide. As another example, butyrylated maltodextrin having a DS of 2.5 is prepared by reacting 10 g of maltodextrin (MW 3000-5000 Da; ˜3 mmols) with 0.32 mols butyric anhydride.

The degree of substitution can influence the hydrophobic character of the polysaccharide. In turn, implants formed from hydrophobic derivatives having a substantial amount of groups having the hydrocarbon segments bonded to the polysaccharide backbone (as exemplified by a high DS) are generally more hydrophobic and can be more resistant to degradation. For example, an implant formed from maltodextrin-butyrate DS1 has a rate of degradation that is faster than an implant formed from maltodextrin-butyrate DS2.

The type of hydrocarbon segment present in the groups pendent from the polysaccharide backbone can also influence the hydrophobic properties of the polymer. In one aspect, the implant is formed using a hydrophobic polysaccharide having pendent groups with hydrocarbon segments being short chain branched alkyl group. Exemplary short chain branched alkyl group are branched C₄-C₁₀ groups. The preparation of a hydrophobic polymer with these types of pendent groups is exemplified by the reaction of maltodextrin with valproic acid/anhydride with maltodextrin (MD-val). The reaction can be carried out to provide a relatively lower degree of substitution of the hydroxyl groups, such as is in the range of 0.5-1.5. Although these polysaccharides have a lower degree of substitution, the short chain branched alkyl group imparts considerable hydrophobic properties to the polysaccharide.

Various synthetic schemes can be used for the preparation of a hydrophobic derivative of a natural degradable polysaccharide. In some modes of preparation, pendent polysaccharide hydroxyl groups are reacted with a compound that includes a hydrocarbon segment and a group that is reactive with the hydroxyl groups. This reaction can provide polysaccharide with pendent groups comprising hydrocarbon segments.

Examples of hydroxyl reactive groups include acetal, carboxyl, anhydride, acid halide, and the like. These groups can be used to form a hydrolytically cleavable covalent bond between the hydrocarbon segment and the polysaccharide backbone. For example, the method can provide a pendent group having a hydrocarbon segment, the pendent group linked to the polysaccharide backbone with a cleavable ester bond. In these aspects, the synthesized hydrophobic derivative of the natural degradable polysaccharide can include chemical linkages that are both enzymatically cleavable (the polymer backbone) and non-enzymatically hydrolytically cleavable (the linkage between the pendent group and the polymer backbone).

Other cleavable chemical linkages (e.g., metabolically cleavable covalent bonds) that can be used to bond the pendent groups to the polysaccharide include carboxylic ester, carbonate, borate, silyl ether, peroxyester groups, disulfide groups, and hydrazone groups. As such, it will be appreciated that degradable polymers herein can include maltodextrin derivatized with silylethers.

Any suitable chemical group can be coupled to the polysaccharide backbone and provide the polysaccharide with hydrophobic properties, wherein the polysaccharide becomes insoluble in water. Specifically, the pendent group can include one or more atoms selected from carbon (C), hydrogen (H), oxygen (O), nitrogen (N), and sulfur (S).

In some aspects, the pendent group includes a hydrocarbon segment that is a linear, branched, or cyclic C₂-C₁₈ group. More specifically the hydrocarbon segment includes a C₂-C₁₀, or a C₄-C₈, linear, branched, or cyclic group. The hydrocarbon segment can be saturated or unsaturated, and can include alkyl groups or aromatic groups, respectively. The hydrocarbon segment can be linked to the polysaccharide chain via a hydrolyzable bond or a non-hydrolyzable bond.

Degradable polymers of the invention can specifically include polysaccharides such as those described in U.S. Publ. Pat. Application No. 2005/0255142, 2007/0065481, 2007/0218102, 2007/0224247, 2007/0260054, all of which are herein incorporated by reference in their entirety.

Degradable polymers of the invention can further include collagen/hyaluronic acid polymers.

In some embodiments, polymeric matrices of embodiments herein can include non-degradable polymers. In an embodiment, the non-degradable polymer includes a mixture of different polymers. As used herein, the term “(meth)acrylate”, when used in describing polymers, shall mean the form including the methyl group (methacrylate) or the form without the methyl group (acrylate). Non-degradable polymers of the invention can include a polymer selected from the group consisting of poly(alkyl(meth)acrylates) and poly(aromatic(meth)acrylates), where “(meth)” will be understood by those skilled in the art to include such molecules in either the acrylic and/or methacrylic form (corresponding to the acrylates and/or methacrylates, respectively). An exemplary polymer is poly(n-butyl methacrylate) (pBMA). Examples of suitable polymers also include polymers selected from the group consisting of poly(aryl(meth)acrylates), poly(aralkyl(meth)acrylates), and poly(aryloxyalkyl(meth)acrylates). Examples of suitable polymers also include poly(ethylene-co-vinyl acetate) (pEVA) having vinyl acetate concentrations of between about 10% and about 50% (12%, 14%, 18%, 25%, 33% versions are commercially available), in the form of beads, pellets, granules, etc. The pEVA co-polymers with lower percent vinyl acetate become increasingly insoluble in typical solvents, whereas those with higher percent vinyl acetate become decreasingly durable.

An exemplary non-degradable polymer mixture includes mixtures of pBMA and pEVA. This mixture of polymers can be used with absolute polymer concentrations (i.e., the total combined concentrations of both polymers in the coating material), of between about 0.25 wt. % and about 99 wt. %. This mixture can also be used with individual polymer concentrations in the coating solution of between about 0.05 wt. % and about 99 wt. %. In one embodiment the polymer mixture includes pBMA with a molecular weight of from 100 kilodaltons to 900 kilodaltons and a pEVA copolymer with a vinyl acetate content of from 24 to 36 weight percent. In an embodiment the polymer mixture includes pBMA with a molecular weight of from 200 kilodaltons to 300 kilodaltons and a pEVA copolymer with a vinyl acetate content of from 24 to 36 weight percent. The concentration of the active agent or agents dissolved or suspended in the coating mixture can range from 0.01 to 99 percent, by weight, based on the weight of the final coating material.

Non-degradable polymers can also comprise one or more polymers selected from the group consisting of (i) poly(alkylene-co-alkyl(meth)acrylates, (ii) ethylene copolymers with other alkylenes, (iii) polybutenes, (iv) diolefin derived non-aromatic polymers and copolymers, (v) aromatic group-containing copolymers, and (vi) epichlorohydrin-containing polymers.

Non-degradable polymers can also include those described in U.S. Publ. Pat. App. No. 2007/0026037, entitled “DEVICES, ARTICLES, COATINGS, AND METHODS FOR CONTROLLED ACTIVE AGENT RELEASE OR HEMOCOMPATIBILITY”, the contents of which are herein incorporated by reference in its entirety. As a specific example, non-degradable polymers can include random copolymers of butyl methacrylate-co-acrylamido-methyl-propane sulfonate (BMA-AMPS). In some embodiments, the random copolymer can include AMPS in an amount equal to about 0.5 mol. % to about 40 mol. %.

### Devices

As described above, dehydrated complexes, microparticles including dehydrated complexes, and polymeric matrices including dehydrated complexes and/or microparticles of embodiments herein can be used by themselves as an implant or device. For example, in some embodiments, dehydrated complexes, microparticles including dehydrated complexes, and polymeric matrices can be injected or otherwise administered to a subject. Additionally, dehydrated complexes can be incorporated in an organic solvent comprising a polymer solution and then deposited on a medical device by spray or dip coating or other coating methods.

In addition, dehydrated complexes, microparticles including dehydrated complexes, and polymeric matrices including dehydrated complexes and/or microparticles, can form part of a device having other elements. For example, in some embodiments, dehydrated complexes and/or microparticles including dehydrated complexes, and polymeric matrices can be disposed on a substrate that forms part of a medical device. In this context, exemplary medical devices can include, but are not limited to, vascular devices such as grafts (e.g., abdominal aortic aneurysm grafts, etc.), stents (e.g., self-expanding stents typically made from nitinol, balloon-expanded stents typically prepared from stainless steel, degradable coronary stents, etc.), valves (e.g., polymeric or carbon mechanical valves, tissue valves, valve designs including percutaneous, sewing cuff, and the like), vena cava filters, aneurysm exclusion devices, artificial hearts, cardiac jackets, and heart assist devices (including left ventricle assist devices), implantable defibrillators, electro-stimulation devices and leads (including pacemakers, lead adapters and lead connectors), implanted medical device power supplies (e.g., batteries, etc.), peripheral cardiovascular devices, atrial septal defect closures, left atrial appendage filters, valve annuloplasty devices (e.g., annuloplasty rings), mitral valve repair devices, vascular intervention devices, ventricular assist pumps, and vascular access devices (including parenteral feeding catheters, vascular access ports, central venous access catheters); surgical devices such as sutures of all types, staples, anastomosis devices (including anastomotic closures), suture anchors, hemostatic barriers, screws, plates, clips, vascular implants, tissue scaffolds, cerebro-spinal fluid shunts, shunts for hydrocephalus, orthopedic devices such as joint implants, acetabular cups, patellar buttons, bone repair/augmentation devices, spinal devices (e.g., vertebral disks and the like), bone pins, cartilage repair devices, and artificial tendons; dental devices such as dental implants and dental fracture repair devices; drug delivery devices such as drug delivery pumps, intravitreal drug delivery devices; ophthalmic devices including orbital implants, glaucoma drain shunts and intraocular lenses; urological devices such as penile devices (e.g., impotence implants), sphincter, urethral, prostate, and bladder devices (e.g., incontinence devices, benign prostate hyperplasia management devices, prostate cancer implants, etc.), synthetic prostheses such as breast prostheses and artificial organs (e.g., pancreas, liver, lungs, heart, etc.); neurological devices such as neurostimulators, neurological catheters, neurovascular balloon catheters, neuro-aneurysm treatment coils, and neuropatches; biosensor devices including glucose sensors, cardiac sensors, intra-arterial blood gas sensors; oncological implants; and pain management implants.

In some aspects, embodiments of the invention can include and be utilized in conjunction with ophthalmic devices. Suitable ophthalmic devices in accordance with these aspects can provide active agent to any desired area of the eye. In some aspects, the devices can be utilized to deliver active agent to an anterior segment of the eye (in front of the lens), and/or a posterior segment of the eye (behind the lens). Suitable ophthalmic devices can also be utilized to provide active agent to tissues in proximity to the eye, when desired.

In some aspects, embodiments of the invention can be utilized in conjunction with ophthalmic devices configured for placement at an external or internal site of the eye. Suitable external devices can be configured for topical administration of active agent. Such external devices can reside on an external surface of the eye, such as the cornea (for example, contact lenses) or bulbar conjunctiva. In some embodiments, suitable external devices can reside in proximity to an external surface of the eye.

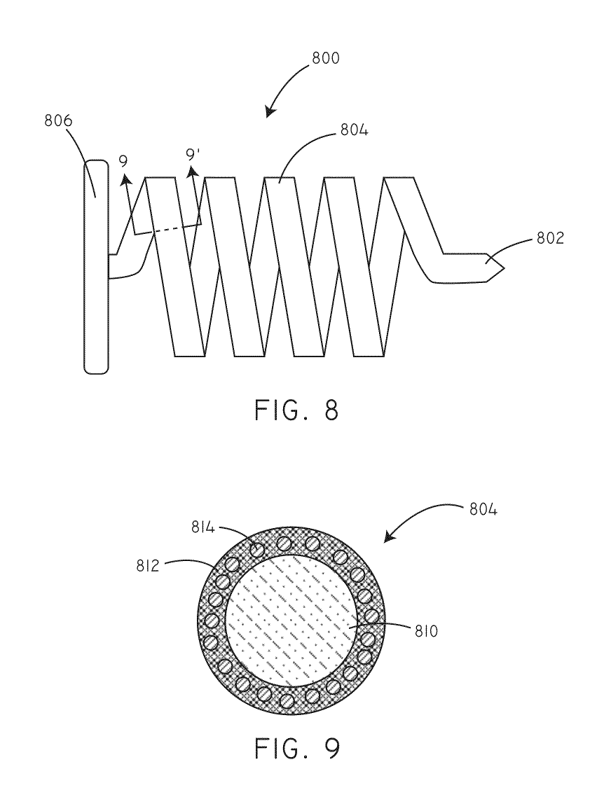

Referring now to FIG. 8, a schematic view is shown of an exemplary medical device 800 in accordance with an embodiment of the invention. In this embodiment, the medical device 800 is an eye screw or eye coil. However, it will be appreciated that other types of medical device are also included within the scope herein. Further examples of medical devices are described below. The medical device 800 includes a tip 802, a coiled body 804, and a cap member 806.

Referring now to FIG. 9, a cross-sectional view of the medical device 300 of FIG. 8 is shown as taken along line 9-9′ of FIG. 8. In this view, an elution control layer 812 is disposed on a substrate 810. The elution control layer 812 can include dehydrated complexes 814 as described herein along with, optionally, one or more other components such as a polymeric matrix including degradable and/or non-degradable polymers. The substrate 810 can include various materials, including but not limited to, metals, ceramics, polymers, glasses, and the like.

The present invention may be better understood with reference to the following examples. These examples are intended to be representative of specific embodiments of the invention, and are not intended as limiting the scope of the invention.

### EXAMPLES

### Example 1

### siRNA/DOTAP Liposomes and siRNA/N-ter Complexes with Saccharide Protectant

Anti-luciferase siRNA and non-targeting control siRNA were obtained from Qiagen (Alameda, Calif.). A series of 50 μl-samples of complexes including siRNA and either DOTAP or N-TER (Sigma-Aldrich, St. Louis, Mo.) were prepared with 1.5 μl siRNA at 20 nM (0.43 μg) per sample. The reagents were added in bulk amounts and the resulting solution was then aliquoted in 50-μl samples.

a) siRNA/N-TER Particle Formation:

For each sample, 1.5 μl siRNA was diluted in 45 μl N-TER buffer (Sigma-Aldrich, St. Louis, Mo.). 3.75 μl N-TER solution (Sigma-Aldrich, St. Louis, Mo.) was added and vortexed. 50 μl glycogen (Shellfish Derived, MP Biomedicals, MW unspecified—typically˜1,000,000 Da) in distilled deionized water at 25 mg/ml or 50 mg/ml was added to the resulting complexes. The mixtures were then lyophilized using a bench-top lyophilizer. Lyophilized samples with glycogen were dispersed in dichloromethane (DCM) and then vacuum dried to remove solvent.

b) siRNA/DOTAP/Cholesterol Particle Formation:

DOTAP was obtained from Avanti Polar Lipids (Alabaster, Ala.) and cholesterol was obtained from Sigma-Aldrich (St. Louis, Mo.). For each sample, 1.5 μl siRNA at 20 uM was diluted in 45 μl distilled deionized water. 4.2 μl of a 1 mg/ml DOTAP/Cholesterol solution in ethanol at 9:1 ratio of DOTAP to cholesterol was added and vortexed well. 50 μl glycogen in distilled deionized water at 25 mg/ml or 50 mg/ml was added to the resulting liposome complexes. The mixtures were then lyophilized using a bench-top lyophilizer yielding a fine particulate solid that could easily be suspended in organic solvents. Lyophilized samples with glycogen were dispersed in dichloromethane (DCM) and then vacuum dried to remove solvent.

### c) Controls:

Control samples were prepared by combining siRNA with DOTAP or N-TER without adding excipients such as glycogen. Alternatively, glycogen was added to a freshly prepared siRNA complex with DOTAP or N-ter without subsequent lyophilization.

### Testing Procedure:

To the: 1.) lyophilized, 2.) lyophilized and solvent suspended and dried samples, and 3.) liquid controls, cell media with 5 μg/ml doxycycline (dox) was added to an end volume of 600 μl (siRNA at 50 nM concentration). 100 μl was added to 4 wells of 96-well plate that was seeded with HR5CL11 cells (10,000 cells per well) 24 hours prior to the transfection. The remaining solution was diluted with media obtaining siRNA at 25 nM and again 100 μl was put in 4 wells. For N-ter containing complexes, the cell media consisted of DMEM 10% w/v FBS and the media with transfection complexes was left for 24 hours. For DOTAP/siRNA complexes DMEM/5 μg/ml dox was used without FBS. After 3 hours of incubation the cell media was replaced with fresh DMEM/10% FBS/5 μg/ml dox and further incubated for 24 hours.

The cells were then incubated with Cell Titer Blue (Promega) diluted in DMEM/10% FBS as per manufacturer recommendation for 1.5 hours to assess any toxicity. All media was removed and cells were lysed using Glo Lysis Buffer (Promega). Luciferase content was measured by luminescence using the Bright-Glo luciferase assay (Promega). The raw data obtained from luciferase assay was normalized against the toxicity data by division and subsequent multiplication by 5000. Gene knock-down is expressed as 100%-RLU_{luc}/RLU_{control}*100%. RLU=relative light units.

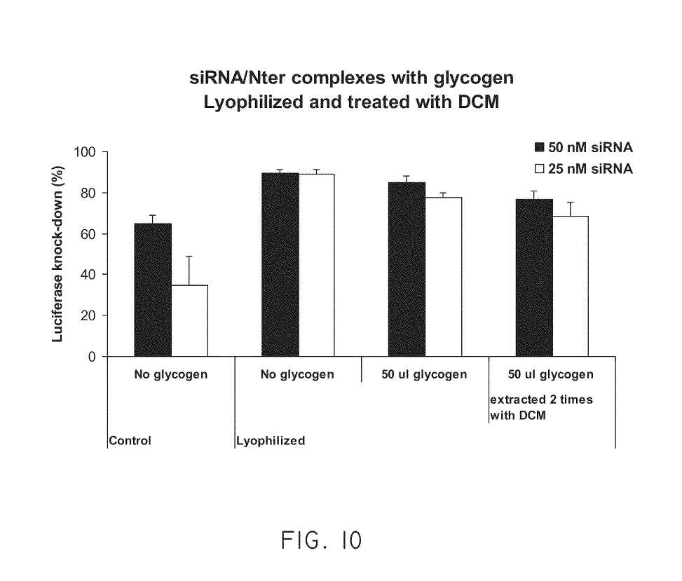

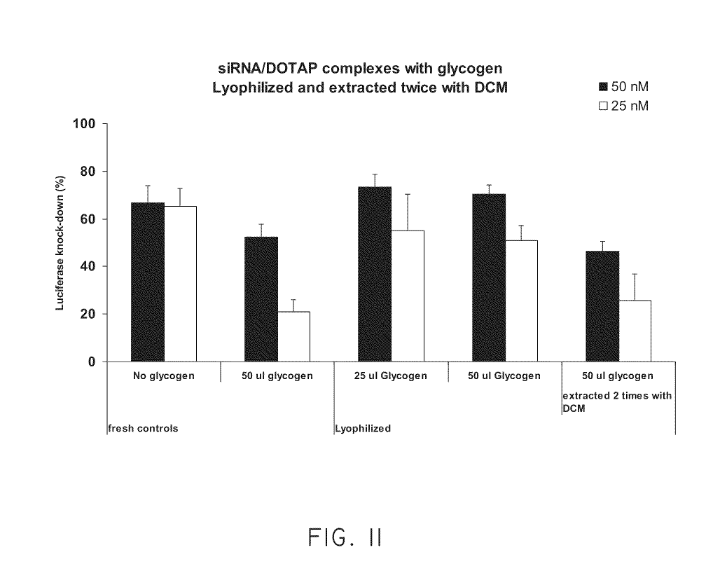

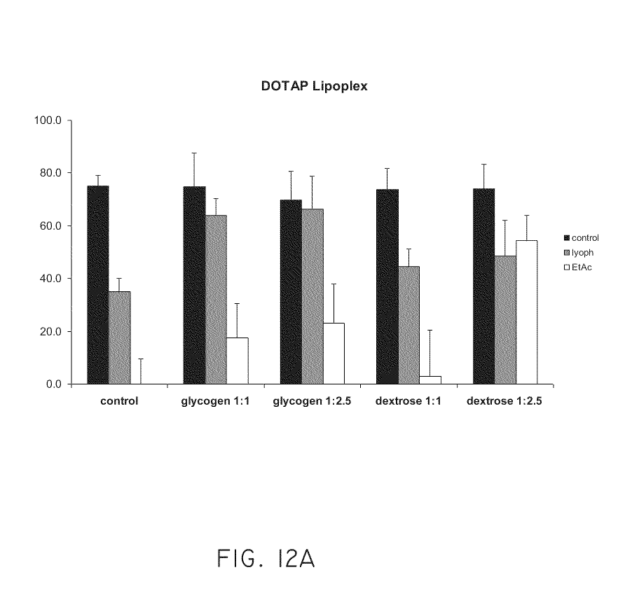

The results are shown in FIGS. 10-11.

### Example 2

### Optimization of siRNA/DOTAP Lipoplex or Liposome Activity with Glycogen

Liposomes were prepared as follows. 17.5 μl siRNA 20 uM was diluted in 72.5 μl distilled deionized water. 10 μl DOTAP 5 mg/ml in EtOH was added, mixed and sonicated.

Lipoplexes were prepared as follows. DOTAP in ethanol was evaporated in a rotovap device under vacuum to form a thin film. The DOTAP film was then dissolved in water at 1 mg/ml, sonicated and then filtered through a 0.2 μm vacuum filter. 17.5 μl 20 uM siRNA was diluted in 32.5 μl distilled deionized water. 50 μl DOTAP at 1 mg/ml in distilled deionized water was added and mixed well.

Glycogen or dextrose solutions in distilled deionized water were added to yield 1:1 or 1:2.5 wt/wt ratios of nucleic acid/transfection agent complexes versus saccharide protectant. The resulting mixtures were lyophilized and resuspended in 250 μl of ethyl acetate. To ensure thorough suspension, the mixture was sonicated. Then the solvent was removed in vacuum. The solids were redissolved in serum-free DMEM yielding siRNA concentrations of 50 nM and put on HR5CL11 cells to test for activity. 100 μl was added to 3 wells of a 96-well plate that was seeded with HR5CL11 cells at 10,000 cells/well 24 hours prior to the transfection. After 3 hours of incubation the cell media was replaced with fresh DMEM/10% FBS/5 μg/ml dox and further incubated for 24 hours.

The cells were then incubated with Cell Titer Blue for 1.5 hours to assess any toxicity. All media was removed and cells were lysed using Glo lysis buffer. Luciferase content was measured by luminescence using the Bright-Glo luciferase assay. The raw data obtained from luciferase assay was normalized against the toxicity data by division and subsequent multiplication by 5000. Gene knock-down is expressed as 100%-RLU_{luc}/RLU_{control}*100%. RLU=relative light units.

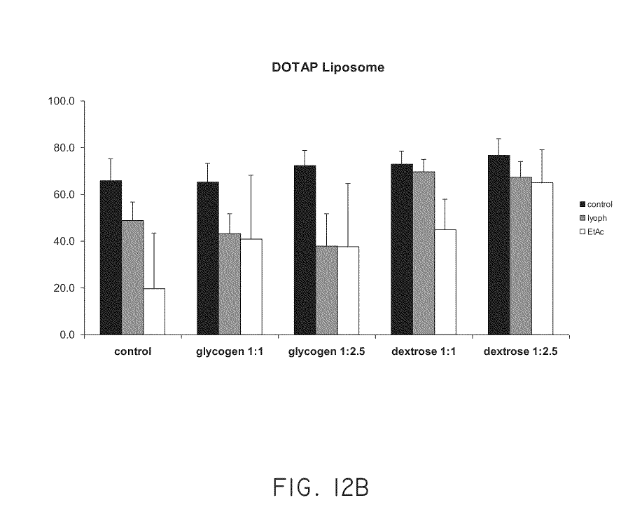

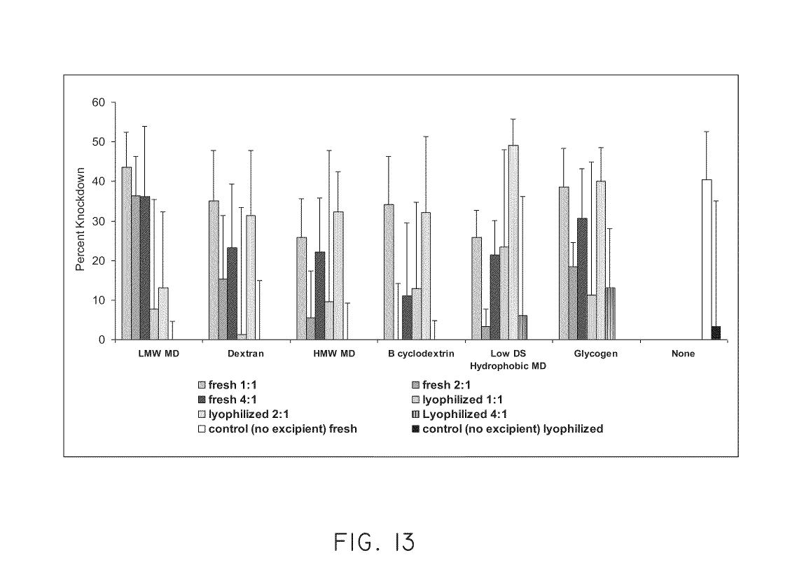

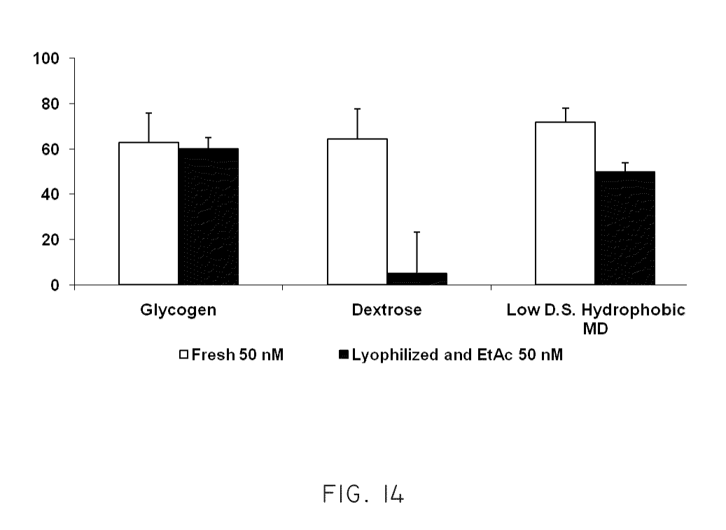

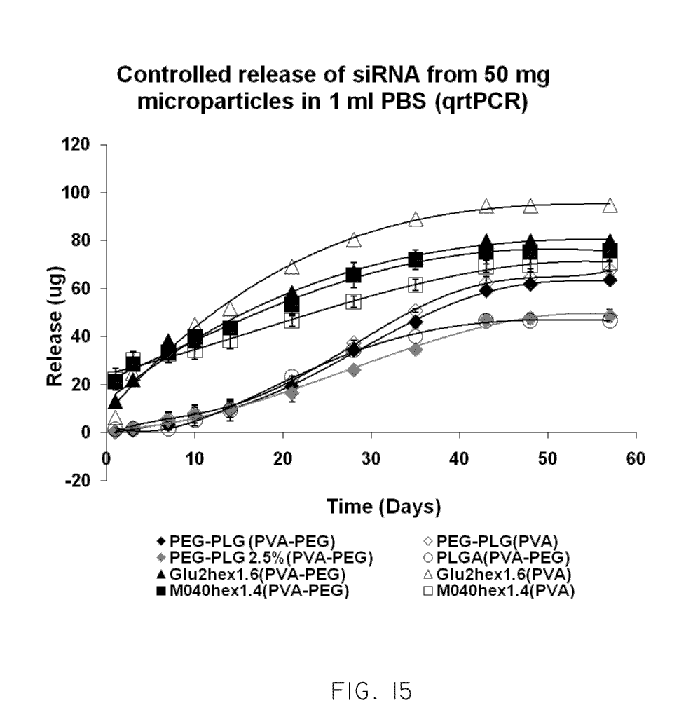

The results are shown in FIGS. 12A-12B.

### Example 3

### Effect of Different Polysaccharides on Activity after Lyophilization and Solvent Exposure

The following polysaccharides were used in this example:

1. Low molecular weight maltodextrin (LMW MD) (<30,000 Da (as fractionated), GPC, Muscatine, Iowa)

2. Dextran 40 kDa (Sigma, St. Louis, Mo.)

3. High molecular weight maltodextrin (HMW MD) 320 kDa (Roquette, France)

4. β-cyclodextrin (Alfa Aesar, Ward Hill, Mass.)

5. Hydrophobically modified maltodextrin (Low DS Hydrophobic MD) 320 kDa was prepared as described in US Patent Application 2007/0260054 at a low degree of substitution (modified with hexanoate at D.S. 0.1) such that it remained soluble in water.

6. Shellfish derived glycogen (MW typically around 1,000,000) (MP Biomedicals).

Using 10 ml siRNA 100 μg/ml, and 10 ml DOTAP 1 mg/ml in water, the procedure described in Example 2 was followed to form lipoplexes. To 1 ml of lipoplex (50 μg siRNA, 500 μg DOTAP) 100 μl, 200 μl or 400 μl of a saccharide solution at 5 mg/ml in water was added to obtain 1:1, 2:1 or 4:1 excipient:complex ratios. The resulting solutions were divided in two equal volumes. One half was lyophilized and resuspended in ethyl acetate. The other part was kept at 4° C. Distilled deionized water was then added to all samples to obtain an siRNA concentration of 2.5 uM.

The samples were diluted in serum-free DMEM until siRNA was at 50 nM concentration. 100 μl was added to 3 wells of a 96-well plate that was seeded with HR5CL11 cells at 10⁵ cell/ml and incubated for 24 hours prior to the transfection. After 3 hours of incubation the cell media was replaced with fresh DMEM/10% FBS/5μg/ml dox and further incubated for 24 hours.

The cells were then incubated with Cell Titre Blue for 1.5 hours to assess any toxicity. All media was removed and cells were lysed using Glo lysis buffer. Luciferase content was measured by luminescence using the Bright-Glo luciferase assay. The raw data obtained from luciferase assay was normalized against the toxicity data by division and subsequent multiplication by 5000. Gene knock-down is expressed as 100%-RLU_{luc}/RLU_{control}*100%. RLU=relative light units.

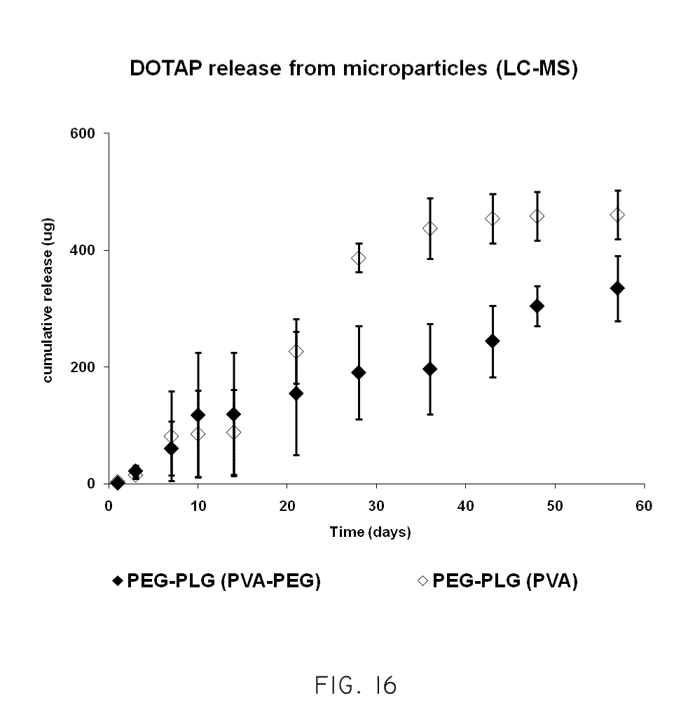

The results are shown in FIG. 13.

### Example 4

### Scale-Up siRNA/DOTAP Lipoplex with Polysaccharides

siRNA/DOTAP complexes were made in 125 μg siRNA quantities with 1.25 mg DOTAP, using DOTAP micelles in water at 1 mg/ml to form lipoplexes. Dextrose (monosaccharide), glycogen (Shellfish derived, as described in Example 3), or water soluble hydrophobically modified maltodextrin (LOW DS Hydrophobic MD as described in Example 3) were dissolved in distilled deionized water at 2.5 mg/ml and added as saccharide protectants to the siRNA/DOTAP complexes at 1:2.9 wt ratio (complex:saccharide). From the batches samples were taken, lyophilized and resuspended in ethyl acetate. The samples were dried in vacuum and subsequently redissolved in serum-free DMEM. The samples were further diluted in DMEM until siRNA was at 50 nM concentration. 100 μl was added to 3 wells of a 96-well plate that was seeded with HR5CL11 cells at 10⁵ cell/ml and incubated for 24 hours prior to the transfection. After 3 hours of incubation the cell media was replaced with fresh DMEM/10% FBS/5 μg/ml doxycycline and further incubated for 24 hours.

The cells were then incubated with Cell Titer Blue for 1.5 hours to assess any toxicity. All media was removed and cells were lysed using Glo lysis buffer. Luciferase content was measured by luminescence using the Bright-Glo luciferase assay. The raw data obtained from luciferase assay was normalized against the toxicity data by division and subsequent multiplication by 5000. Gene knock-down is expressed as 100%-RLU_{luc}/RLU_{control}*100%. RLU=relative light units.

The results are shown in FIG. 14.

### Example 5

### Microparticles Containing siRNA/DOTAP/Glycogen Complexes

Microparticles have been prepared based on solid in oil/water single emulsion process. In preparation of these formulations the following batches have been made 17 times with anti-luciferase siRNA and 9 times with scrambled (control) siRNA: 88.0 μl siRNA at 1 mM (1.25 mg) was diluted in 12.5 ml distilled deionized water. To the solution 12.5 ml of DOTAP at 1 mg/ml (1:10 ratio w/w siRNA, 12.5 mg) was added and mixed by pipetting up and down multiple times. Then 36.3 mg glycogen was added as 7.25 ml of a 5 mg/ml in distilled deionized water. The solutions were freeze-dried in a temperature controlled lyophilizer.

The abbreviations “Glu2”, and “MO40” refer to maltodextrin polymers having an approximate molecular weight as shown in the table. The abbreviations “Hex” and “Pro” refer to hexanoate and propanoate pendant groups on the maltodextrin polymers. The number after “Hex” and “Pro” refers to the degree of substitution on the polymers.

TABLE 1

| Designation | Maltodextrin M_{w} | Pendent Hydrophobic Group |
|---|---|---|
| Glu2-Hex-x | 330 kDa | Hex = hexanoate |
| Glu2-Pro-x | 330 kDa | Pro = Propanoate |
| MO40-Hex-x | 50 kDa | Hex = hexanoate |

X = degree of substitution (DS); final MW of polymer depends on DS.

The following solutions were prepared:

20% w/w polymer solutions in ethyl acetate by weight. Hydrophobically modified maltodextrins were prepared as described in US Patent # 2007/0260054.

A) The polymers used were:

a. maltodextran modified with hexanoate, D.S. 1.6 (“Glu2hex1.6”);

b. maltodextran modified with hexanoate, D.S. 1.4 (“M040hex1.4”);

c. Poly(lactide-co-glycolide) (PLGA), 50/50 wt. % lactide/glycolide, intrinsic viscosity=0.35, (“DLG 3.5E”, obtained from Lakeshore Biomaterials, Birmingham, Ala.); or

d. Di-block copolymer of methoxy-polyethyleneglycol (Mw 1500 Da) and poly(lactide-co-glycolide) (PLGA block 65/35 wt. % lactide/glycolide), intrinsic viscosity=0.49 (“PEG-PLG 65/35 (lactide/glycolide)”, Lakeshore Biomaterials, Birmingham, Ala.), PEG comprising approximately 2-3 wt. % of di-block copolymer.

B) Continuous phase:

a. 2% PVA (Amsresco, 35-50 kDa) (polyvinyl alcohol) in distilled deionized water; or

b. 0.5% PVA/4% PEG 20 kDa in distilled deionized water (PVA-PEG).

Both solutions were saturated with ethyl acetate.

Lyophilized siRNA (either luciferase or non-targeting control), DOTAP and glycogen preparations were combined to yield 100 mg of material containing 2.5 mg siRNA, 25 mg DOTAP and 72.5 mg glycogen. 2.5 grams of polymer solution (500 mg polymer) was added to lyophilized siRNA/DOTAP/glycogen. The solids were dispersed using an IKA-25T probe at setting “3” for 30 seconds. The mixture was emulsified in 150 ml continuous phase for 15 seconds (Silverson, 2-arm probe, 1000 rpm). To harden, the emulsion was poured in 850 ml distilled deionized water and stirred for 30 minutes. The particles were filtered through a stack of 125 um and 20 um filters. The particles 20-125 and <20 um (if present) were collected and lyophilized. The filtrate was spun to collect particles <20 um. 400 ml of the supernatant was lyophilized to determine free siRNA.

The following batches were made:

TABLE 2

| | | | | Load |
|---|---|---|---|---|
| | | Hardening | | Mass % Solids to |
| Batch | Polymer | Bath | SiRNA | Polymer |
| 1 | PEG-PLG | PVA-PEG | Lu | 20% |
| 2 | PEG-PLG | PVA | Lu | 20% |
| 3 | Hydrophobic | PVA-PEG | Lu | 20% |
| | MD A | | | |
| 4 | Glu2hex1.6 | PVA | Lu | 20% |
| 5 | PLGA | PVA-PEG | Lu | 20% |
| 6 | PLGA | PVA | Lu | 20% |
| 7 | PEG-PLG | PVA-PEG | Lu | 10% |
| 8 | PEG-PLG | PVA-PEG | Control | 20% |
| 9 | PLGA | PVA-PEG | Control | 20% |
| 10 | Glu2hex1.6 | PVA-PEG | Control | 20% |
| 11 | M040hex1.4 | PVA-PEG | Lu | 20% |
| 12 | M040hex1.4 | PVA | Lu | 20% |
| 13 | PEG-PLG | PVA-PEG | Control | 10% |
| 14 | M040hex1.4 | PVA-PEG | Control | 20% |

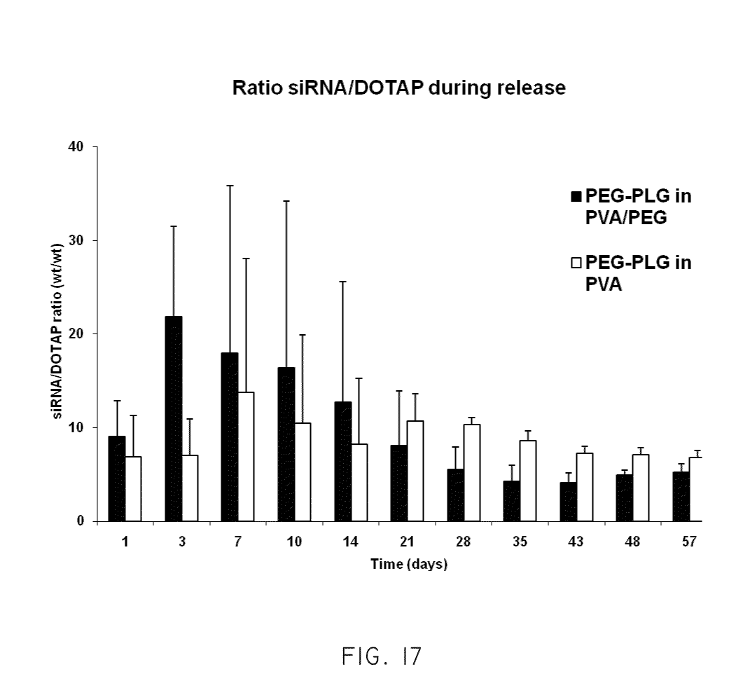

To study the controlled release, about 50 mg per microparticle formulation was put in 1 ml PBS and left at 37° C. At specific time intervals the buffer was replaced with fresh PBS. Release of siRNA was measured using qrtPCR. siRNA was reverse transcribed to cDNA using a MicroRNA Reverse Transcription Kit (Applied Biosystems) with an siRNA specific stem loop primer. cDNA was then assessed by real time PCR using TAQMAN primers and probes specific for siRNA sequence (Reagents and equipment from Applied Biosytems). Results are shown in FIG. 15.

Additionally, controlled release of DOTAP from batches 1 and 2 was analyzed by determining DOTAP concentration using LC/MS. The ratio of DOTAP to siRNA was determined by dividing the DOTAP concentration at each release time point by the concentration of siRNA at each time point. Results are shown in FIGS. 16 and 17.

To study bioactivity of release DOTAP/siRNA he elution buffer was diluted with DMEM at 1:1 or 1:5 ratios. 100 μl of diluted complex was added in triplicate to a 96-well plate that was seeded with HR5CL11 cells at 10,000 cells per well 24 hours prior to the transfection. After 3 hours of incubation the cell media was replaced with fresh DMEM/10% FBS/5 μg/ml dox and further incubated for 24 hours.

The cells were then incubated with Cell Titer Blue for 1.5 hours to assess any toxicity. All media was removed and cells were lysed using Glo lysis buffer. Luciferase content was measured by luminescence using the Bright-Glo luciferase assay.

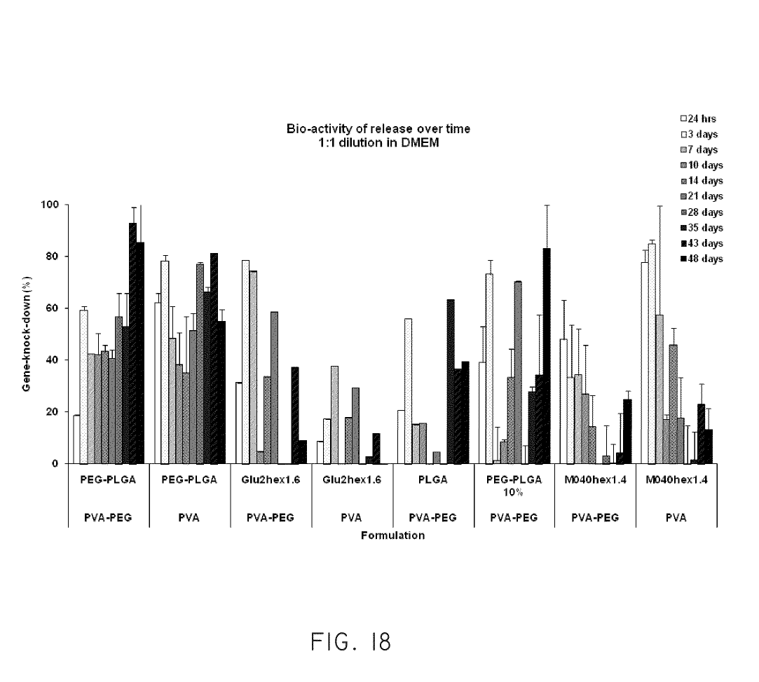

The raw data obtained from luciferase assay was normalized against the toxicity data by division and subsequent multiplication by 5000. Gene knock-down is expressed as 100%-RLU_{luc}/RLU_{control}*100%. RLU=relative light units. Results are shown in FIG. 18.

### Example 6

### Organogels/Terpolymers Containing siRNA/DOTAP/Glycogen Complexes

Lyophilizates with siRNA/DOTAP/glycogen as described in Example 4 were used. Maltodextrin (Glu2) hexanoate with D.S 1.6 (Glu2Hex1.6) and Maltodextrin (Glu2) Propanoate with D.S. 1.6 (Glu2Pro1.7) were prepared as described in US Patent Application # 2007/0260054. Glu2Hex1.6 was then dissolved in benzylbenzoate (BB) at 300 mg/ml or in ethylheptanoate (EH) at 300 mg/ml. Glu-2 pro 1.6 was dissolved at 200 mg/ml or at 300 mg/ml in a mixture of benzylbenzoate and glycofurol (GF) at a ratio of 9:1. All formulations were kept at 55° C. 50 mg of lyophilized siRNA/DOTAP/glycogen was combined with 200 mg of polymer formulations. Four organogel formulations were prepared as described below:

1—Glu2Hex 1.6 in BB at 300 mg/ml

2—Glu2Hex 1.6 in EH at 300 mg/ml

3—Glu2Pro 1.7 in BB/GF at 200 mg/ml

4—Glu2Pro 1.7 in BB/GF at 300 mg/ml

Terpolymers were synthesized as described in US Publ. Pat. App. No. 2009/124535. The following terpolymers were used (DL=DL-lactide =, L=L-lactide, G=glycolide, CL=caprolactone):

8′=dodecanol initiator; 19:31:50 wt. %-DL:G:CL; 4700 Da; 206 poise

22=dodecanol initiator; 19:23:58 wt. %-L:G:CL; 2400 Da; 73 poise

23=PEG350 initiator; 16:14:33 wt. %-DL:G:CL; 5200 Da; 401 poise

8′ was also formulated with 10% benzyl benzoate as a solvent to decrease viscosity.

200 mg of terpolymers were mixed with 50 mg of siRNA/DOTAP/glycogen lyophilizates. The resulting mixtures were spun briefly (10 krpm for 30 seconds).

For controlled release studies, 1 ml of PBS was added to the formulations and left at 37° C. At specific time intervals the buffer was replaced with fresh PBS. Of the original mixture of siRNA/DOTAP/Glycogen a sample was dissolved in water and then diluted in PBS and kept at 37° C. and used as control. Collected elution buffer was diluted with DMEM at 1:15 or 1:150 ratios. 100 μl was added in triplicate to a 96-well plate that was seeded with HR5CL11 cells at 10,000 cells per well 24 hours prior to the transfection. After 3 hours of incubation the cell media was replaced with fresh DMEM/10% FBS/5 μg/ml dox and further incubated for 24 hours.

The cells were then incubated with Cell Titer Blue for 1.5 hours to assess any toxicity. All media was removed and cells were lysed using Glo lysis buffer. Luciferase content was measured by luminescence using the Bright-Glo luciferase assay. The raw data obtained from luciferase assay was normalized against the toxicity data by division and subsequent multiplication by 5000. Gene knock-down is expressed as 100%-RLU_{luc}/RLU_{control}*100%. RLU=relative light units.

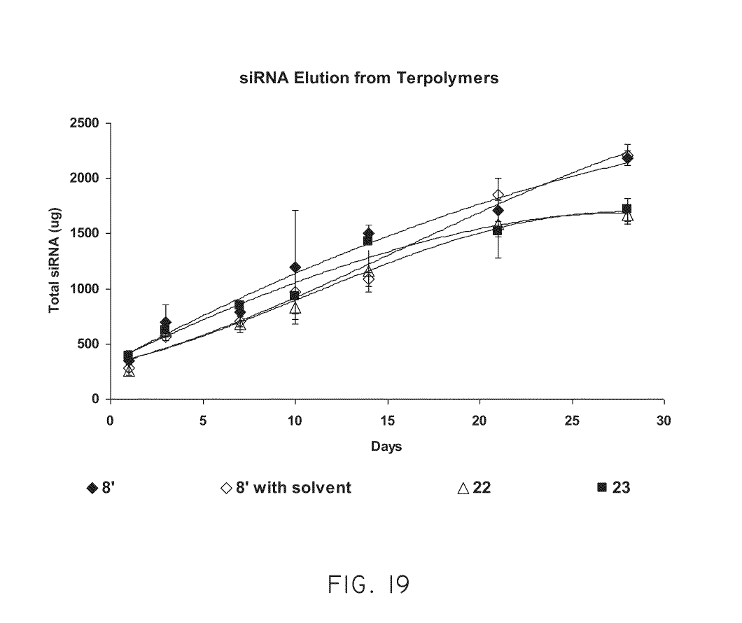

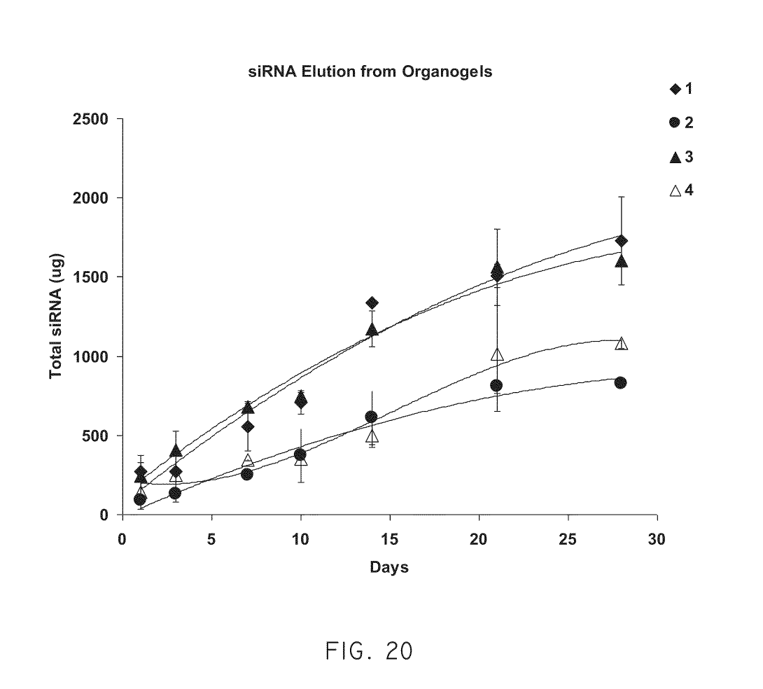

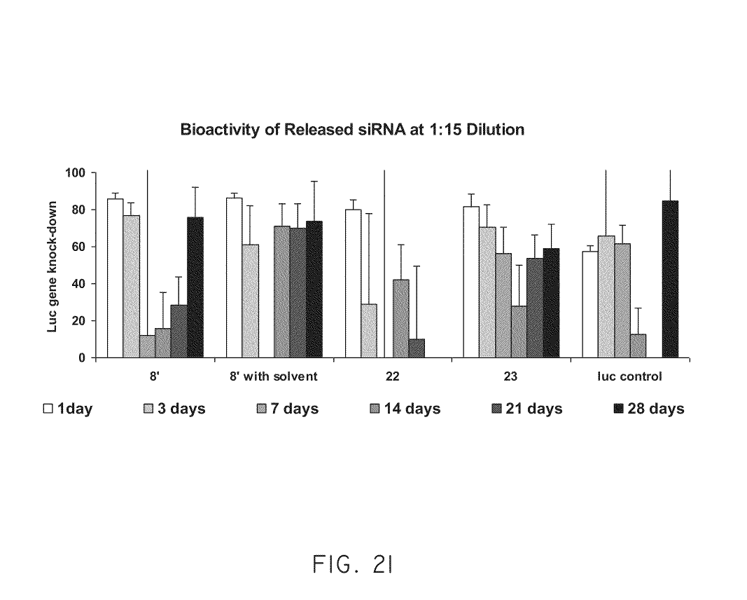

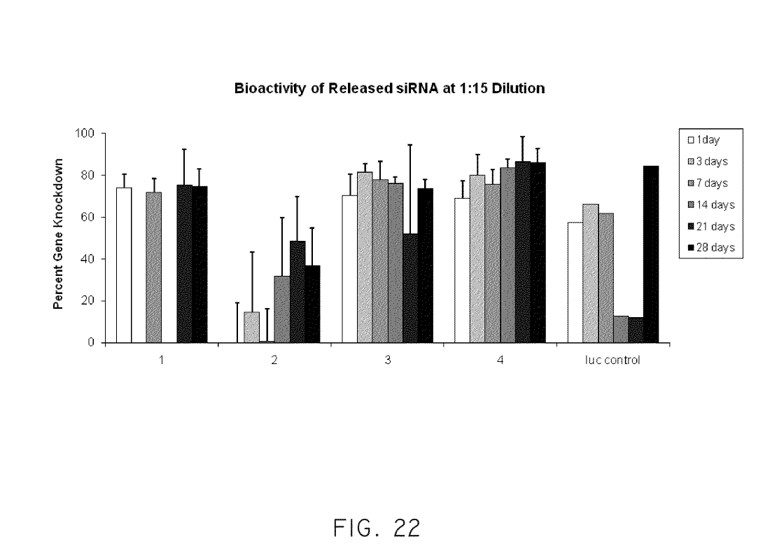

Release of siRNA was measured as in example 5. The results are shown in FIGS. 19 and 20. Bioactivity of released siRNA complexes is shown in FIGS. 21 and 22.

### Example 7

### Glycogen Retains Activity of Cationic Lipid Complexes after Exposure to Solvents at Lower Concentrations than Dextrose

Glycogen (Shellfish Derived, MP Biomedicals, M.W. typically 1,000,000 Da) was obtained from MP Biomedicals, Inc. Dextrose was obtained from Sigma and LIPOFECTAMINE RNAiMax (LFRNAiMax) (cationic lipid) was obtained from Invitrogen. siRNA targeting luciferase or a non-specific control was obtained from Qiagen.

Lipoplexes of siRNA and lipofectamine were formed in water by combining a solution of siRNA with an aqueous solution of LFRNAiMax (1 μl of LFRNAiMAx to 7 pmol siRNA) according to manufacturer's instructions. Glycogen or dextrose was then added to complexes at 10:1, 5:1 or 1:1 mass ratios. As a control water only was added to complexes. Complexes were subsequently lyophilized and then lyophilized powders were dispersed in ethyl acetate using sonication. Ethyl acetate was then stripped from complexes by vacuum drying and complexes were rehydrated and used in cell culture for gene knockdown assays. As controls fresh complexes and lyophilized only complexes were run.

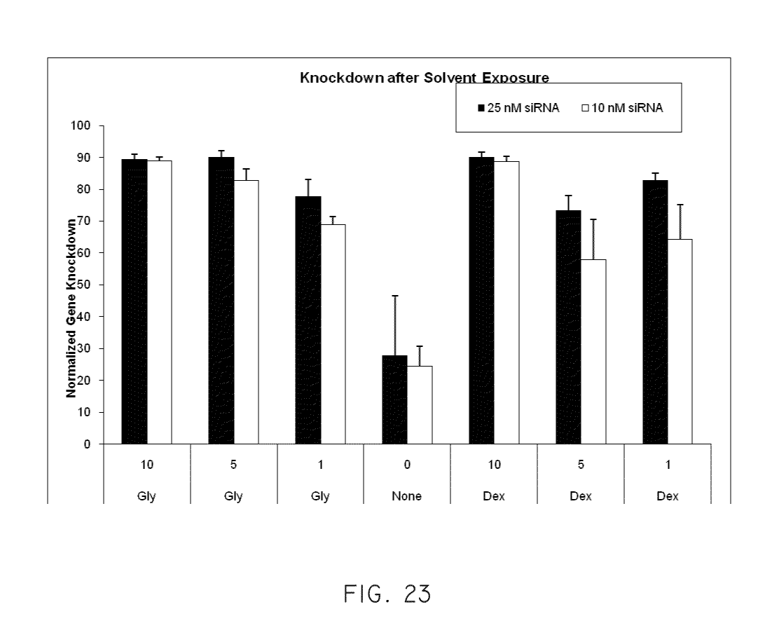

Results for complexes exposed to solvent in the presence of glycogen and dextrose are shown in FIG. 23.

### Further Embodiments:

In various embodiments the invention includes an article for delivering an active agent including a dehydrated complex comprising a nucleic acid and a transfection agent, and a saccharide protectant. In some embodiments, the dehydrated complex can include a lyophilized particulate. In some embodiments, the dehydrated complex can include a spray dried particulate. In some embodiments, the transfection agent can include a lipid transfection agent. In some embodiments, the lipid transfection agent can include a cationic lipid. In some embodiments, the transfection agent can include a lipidoid. In some embodiments, the dehydrated complex can include SNALPs (stable nucleic acid-lipid particles). In some embodiments, the w/w ratio of saccharide protectant to the nucleic acid and cationic lipid in the dehydrated complex less than 5 to 1. In some embodiments the nucleic acid can include siRNA. In some embodiments the saccharide protectant can include a linear polysaccharide. In some embodiments the saccharide protectant can include a branched polysaccharide. In some embodiments the saccharide protectant can include glycogen. In some embodiments the saccharide protectant can include maltodextrin. In some embodiments the saccharide protectant can be derivatized with hydrophobic groups. In some embodiments the saccharide protectant can include maltodextrin derivatized with hydrophobic groups and can have a degree of substitution of less than 0.3, the derivatized maltodextrin being water soluble. In some embodiments the nucleic acid and transfection agent can include a liposome. In some embodiments the nucleic acid and transfection agent can include a lipoplex. In some embodiments the article can further include a first polymeric matrix, the dehydrated complex dispersed within the first polymeric matrix. In some embodiments the first polymeric matrix can include a polymer that is degradable. In some embodiments the first polymeric matrix can include a layer that has been formed by spray coating or dip coating. In some embodiments the first polymeric matrix can include a polymer including one or more subunits selected from the group consisting of lactide, glycolide, caprolactone, polyethyleneglycol, or derivatives thereof. In some embodiments the first polymeric matrix can include a terpolymer. In some embodiments the first polymeric matrix can include a viscous fluid. In some embodiments the first polymeric matrix and the dehydrated complex can be a microparticle. In some embodiments the microparticle can have a diameter from about 1 um to about 150 um. In some embodiments the microparticle can have a diameter from about 20 um to about 80 um. In some embodiments the article can further include a second polymeric matrix, the microparticle disposed within the second polymeric matrix, the second polymeric matrix including a different polymer than the first polymeric matrix.

In various embodiments, the invention includes a method of maintaining the transfection activity of a nucleic acid and transfection agent complex for incorporation in a controlled release formulation comprising combining a nucleic acid, a transfection agent, and a saccharide protectant in an aqueous solution to form an active agent composition; and removing water from the active agent composition to form dehydrated complexes. In various embodiments, removing water can include lyophilizing the active agent composition. In various embodiments, removing water can include spray drying the active agent composition. In various embodiments, the method can further include resuspending the dehydrated complexes in an organic solvent. In various embodiments, the transfection agent can include a lipid transfection agent. In various embodiments, the lipid transfection agent can include a cationic lipid. In various embodiments, the transfection agent can include a lipidoid. In various embodiments, the nucleic acid and transfection agent together can comprise SNALPs (stable nucleic acid-lipid particles). In various embodiments, the w/w ratio of saccharide to the nucleic acid and cationic lipid in the active agent composition is less than 5 to 1. In various embodiments, the nucleic acid can include siRNA. In various embodiments, the saccharide protectant can include a linear polysaccharide. In various embodiments, the saccharide protectant can include a branched polysaccharide. In various embodiments, the saccharide protectant can include glycogen. In various embodiments, the saccharide protectant can include maltodextrin. In various embodiments, the saccharide protectant is derivatized with hydrophobic groups. In various embodiments, the saccharide protectant can include maltodextrin derivatized with hydrophobic groups and can have a degree of substitution of less than 0.3, the derivatized maltodextrin soluble in water. In various embodiments, the nucleic acid and transfection agent can be a liposome. In various embodiments, the nucleic acid and transfection agent can be a lipoplex.

In various embodiments, the invention includes a method of making a controlled release formulation comprising combining a nucleic acid, a transfection agent, and a saccharide in an aqueous solvent to form an active agent composition; processing the active agent composition to remove the aqueous solvent and form dehydrated complexes; and combining the dehydrated complexes with a polymer composition. In various embodiments, the method further includes resuspending the dehydrated complexes in an organic solvent prior to combining the dehydrated complexes with the polymer composition. In various embodiments, the polymer composition includes an organic solvent. In various embodiments, the method further includes processing the dehydrated complexes and polymer composition to form microparticles. In various embodiments, the polymeric composition includes a polymer including one or more subunits selected from the group consisting of lactide, glycolide, polyethylene glycol, and caprolactone, or derivatives thereof. In various embodiments, processing the active agent composition to remove the aqueous solvent can include lyophilizing the active agent composition. In various embodiments, processing the active agent composition to remove the solvent can include spray drying the active agent composition. In various embodiments, the transfection agent can include a lipid transfection agent. In various embodiments, the lipid transfection agent can include a cationic lipid. In various embodiments, the transfection agent can include a lipidoid. In various embodiments, the nucleic acid and transfection agent together can include SNALPs (stable nucleic acid-lipid particles). In various embodiments, the w/w ratio of saccharide to the nucleic acid and cationic lipid in the active agent composition is less than 5 to 1. In various embodiments, the nucleic acid can include siRNA. In various embodiments, the saccharide protectant can include glycogen. In various embodiments, the nucleic acid and transfection agent can include a liposome. In various embodiments, the nucleic acid and transfection agent can include a lipoplex.

1. An article for delivering an active agent comprising:
a dehydrated complex comprising
a nucleic acid and a transfection agent, and
a saccharide protectant.

2. The article of claim 1, the dehydrated complex comprising at least one selected from the group consisting of a lyophilized particulate and a spray dried particulate.

3. The article of claim 1, the transfection agent comprising at least one selected from the group consisting of a lipid transfection agent and a lipidoid.

4. The article of claim 1, the w/w ratio of saccharide protectant to the nucleic acid and cationic lipid in the dehydrated complex less than 5 to 1.

5. The article of claim 1, the nucleic acid comprising siRNA.

6. The article of claim 1, the saccharide protectant comprising at least one selected from the group consisting of a linear polysaccharide and a branched polysaccharide

7. The article of claim 1, the saccharide protectant comprising at least one selected from the group consisting of glycogen and maltodextrin.

8. The article of claim 1, the saccharide protectant derivatized with hydrophobic groups.

9. The article of claim 1, the saccharide protectant comprising maltodextrin derivatized with hydrophobic groups and having a degree of substitution of less than 0.3, the derivatized maltodextrin being water soluble.

10. The article of claim 1, further comprising a first polymeric matrix, the dehydrated complex dispersed within the first polymeric matrix.

11. The article of claim 10, the first polymeric matrix comprising a terpolymer.

12. The article of claim 10, further comprising a second polymeric matrix, the first polymeric matrix and the dehydrated complex forming a microparticle, the microparticle disposed within the second polymeric matrix, the second polymeric matrix comprising a different polymer than the first polymeric matrix.

13. A method of maintaining the transfection activity of a nucleic acid and transfection agent complex for incorporation in a controlled release formulation comprising:
combining a nucleic acid, a transfection agent, and a saccharide protectant in an aqueous solution to form an active agent composition; and
removing water from the active agent composition to form dehydrated complexes.

14. The method of claim 13, wherein removing water comprises at least one selected from the group consisting of lyophilizing the active agent composition and spray drying the active agent composition.

15. The method of claim 13, further comprising resuspending the dehydrated complexes in an organic solvent.

16. The method of claim 13, wherein the w/w ratio of saccharide to the nucleic acid and cationic lipid in the active agent composition is less than 5 to 1.

17. The method of claim 13, the saccharide protectant comprising at least one selected from the group consisting of glycogen and maltodextrin.

18. The method of claim 13, the saccharide protectant derivatized with hydrophobic groups.

19. The method of claim 13, the saccharide protectant comprising maltodextrin derivatized with hydrophobic groups and having a degree of substitution of less than 0.3, the derivatized maltodextrin soluble in water.

20. A method of making a controlled release formulation comprising:
combining a nucleic acid, a transfection agent, and a saccharide in an aqueous solvent to form an active agent composition;
processing the active agent composition to remove the aqueous solvent and form dehydrated complexes;
resuspending the dehydrated complexes in an organic solvent; and
combining the dehydrated complexes with a polymer composition.
